(19) United States
(12) Patent Application Publication
(10) Pub. No.: US 2024/0415949 A1
(43) Pub. Date: Dec. 19, 2024

(54) MUCOSAL VACCINE, METHODS OF USE AND ADMINISTRATION THEREOF

(71) Applicant: Jiangsu Recbio Technology Co., Ltd., Taizhou (CN)

(72) Inventor: Kejian Yang, Merritt Island, FL (US)

(73) Assignee: Jiangsu Recbio Technology Co., Ltd., Taizhou (CN)

(21) Appl. No.: 18/702,695

(22) PCT Filed: Oct. 17, 2022
(86) PCT No.: PCT/US2022/078191

§ 371 Date: Apr. 18, 2024

Related U.S. Application Data
(60) Provisional application No. 63/262,661, filed on Oct. 18, 2021.

(51) Int. Cl. A61K 39/12; A61K 39/00; A61P 31/14

(52) U.S. Cl. CPC A61K 39/12; A61P 31/14; A61K 2039/53; A61K 2039/543; A61K 2039/55561; A61K 2039/55572; A61K 2039/57; C12N 2760/18534

(57) ABSTRACT

A method of vaccinating a subject against a respiratory viral infection comprises concurrently or separately administering to a subject in need thereof one of more polynucleotide vector(s) encoding a respiratory virus antigen and one or more aerosolized respiratory virus antigen(s) to induce a mucosal immune response in the subject against an infection by respiratory viral infection. In another aspect, the present application provides a mucosal vaccine kit comprising one of more polynucleotide vector(s) encoding a respiratory virus antigen, one or more aerosolized respiratory virus antigen(s).

This application is the U.S. national phase of International Patent Application No. PCT/US22/78191, filed Oct. 17, 2022, which claims priority from U.S. Provisional Application No. 63/262,661, filed Oct. 18, 2021, which is incorporated herein by reference.

### REFERENCE TO A SEQUENCE LISTING

This application incorporates by reference the Sequence Listing submitted in Computer Readable Form as file NP2024TC1490_SEQ.xml, created on Apr. 15, 2024 and containing 19,882 bytes.

### FIELD

This application generally relates to pharmaceutical compositions for vaccinal immunity and, in particular, to compositions and methods for inducing mucosal immune response to a pathogen.

### BACKGROUND

Mucosal surfaces are enormous surface areas that are vulnerable to infection by pathogenic microorganisms. Mucosal immune responses are most efficiently induced by the administration of vaccines onto mucosal surfaces, whereas injected vaccines are generally poor inducers of mucosal immunity and are therefore less effective against infection at mucosal surfaces. The dose of mucosal vaccine that actually enters the body cannot be accurately measured because antibodies in mucosal secretions are difficult to capture and quantitate, and recovery and functional testing of mucosal T cells is labour intensive and technically challenging. As a result, only a few mucosal vaccines have been approved for human use in the United States or elsewhere.

There remains a need for new mucosal vaccines and methods of use thereof.

### SUMMARY

An aspect of this application is a method for treating or preventing symptoms of a respiratory pathogen infection in a subject, comprising the steps of (1) administering to the subject an effective amount of a priming composition comprising a nucleic acid-based expression system encoding a protein antigen of the respiratory pathogen; and (2) administering to the subject an effective amount of a boosting composition comprising the protein antigen of the respiratory pathogen, wherein the priming composition is administered to the subject non-mucosally, and wherein the boosting composition is administered mucosally. In certain embodiments, the respiratory pathogen is respiratory syncytial virus (RSV).

Another aspect of this application is a mucosal vaccine kit, comprising: (a) a polynucleotide vector comprising a respiratory virus gene operatively linked to a promoter for expressing a respiratory pathogen protein encoded by the gene, wherein the priming composition is formulated for intramuscular injection, and wherein the respiratory virus is RSV and the respiratory pathogen protein is an RSV protein; (b) one or more boosting composition(s), wherein each of the one or more boosting compositions comprises a respiratory pathogen protein and one or more adjuvants, wherein the boosting composition(s) are formulated for intranasal administration, and wherein the respiratory pathogen protein is an RSV protein; and (c) one or more pharmaceutically acceptable carrier(s), wherein the polynucleotide vector in the priming composition encodes the same RSV protein present in the one or more boosting compositions.

Another aspect of the application is an RSV mucosal vaccine, comprising: (a) an effective amount of RSV pre-F protein; and (b) mucosal vaccine adjuvants comprising one or more selected from the group consisting of CpG, with or without MPL, wherein the vaccine is formulated for intranasal administration.

Another aspect of the present application relates to a vaccination kit for RSV, comprising a vaccine composition comprising (a) RSV pre-F protein and (b) an adjuvant, wherein the vaccine (a), optionally formulated in a boosting composition for intranasal delivery; (c) CpG 7909 alone or CpG 7909 and monophosphoryl lipid A (MPL), and (d) one or more pharmaceutically acceptable carriers.

### BRIEF DESCRIPTION OF THE DRAWINGS

FIG. 1 shows a summary of the animal groups in an exemplary RSV vaccine study.

FIG. 2 is a table summarizing the immunization and challenge study design in the RSV vaccine study.

FIG. 3 shows the immunogenicity of the RSV vaccine an RSV vaccine study before RSV challenge. Panel A shows serum IgG antibody levels in each animal group. Panel B shows peak IgG responses in each animal group.

FIG. 4 shows RSV virus titers 5 day after intranasal live RSV challenge. Panel A shows lung virus titers in each animal group. Panel B shows nasal virus titers in each animal group.

FIG. 5 shows neutralizing antibody (Nab) titers at Day 0, Day 28 and Day 49 in each animal group.

FIG. 6 shows histopathology scores 5 days after intranasal RSV challenge in each animal group, including histology scores for peribronchiolitis (panel A), perivasculitis (panel B), interstitial pneumonia (panel C) and alveolitis (panel D).

FIG. 7 shows another perspective of the histopathology scores in FIG. 6 for each animal group with respect to peribronchiolitis, perivasculitis, interstitial pneumonia and alveolitis.

FIG. 8 shows nasal and lung IgA and IgG responses after intranasal RSV challenge. Panel A shows nasal IgA levels in each animal group. Panel B shows lung IgA levels in each animal group. Panel C shows nasal IgG levels in each animal group. Panel D shows lung IgG levels in each animal group.

FIG. 9 shows mRNA expression levels of RSV NS1 in lung following RSV challenge.

FIG. 10 shows a vector map for NTV8385-VA1-RSV-F-A2, DS-Cavl.

FIG. 11 shows the nucleotide sequence of NTV8385-VA1-RSV-F-A2, DS-Cavl (SEQ ID NO: 4).

FIG. 12 shows codon optimized nucleotide sequence of RSV-F-A2 (SEQ ID NO: 7).

While the present disclosure will now be described in detail, and it is done so in connection with the illustrative embodiments, it is not limited by the particular embodiments illustrated in the figures and the appended claims.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Applicant has unexpected discovered that the foregoing protocol is particularly effective for inducing a prophylactic immune response in a subject with or without pre-existing immunity to a respiratory pathogen, such as RSV.

The mucosal vaccines of the present application induce a synergistic immune response comprised of humoral, T cell, and mucosal immunity. Specifically, the vaccine stimulates high titers of nasal and lung antigen-specific IgG and IgA antibodies, a Th1-biased immune response, and high titers of serum antigen-specific IgG. Thus, the present application provides a method for eliciting prophylactic and/or therapeutic immune responses against respiratory pathogens, such as RSV, in a subject, such as a mammal or human.

The vaccine compositions and methods of the present application provide a means to specifically target the most common entry portal for microorganisms, the mucosal surfaces of the body. The invention provides more effective vaccine compositions by directly delivering the immunogenic components, such as protein antigen, to mucosal surfaces. By employing appropriate immunization regimens as described herein, immune responses can be tailored to provide more effective and specific vaccines. In preferred embodiments, administration of the vaccine compositions generates a balanced or T helper 1 (Th1)-biased immune response that also includes robust antibody responses, CTL generation and Th1-type cytokine production, and local immunity at mucosal sites.

As shown in the Examples, administration of the priming and boosting compositions of the present application results in not only an induction of a CTL response, but also in a robust mucosal immune response. Specifically, viral shedding is reduced or completely prevented in a subject infected with the respiratory pathogen subsequent to administration of the priming and boost compositions as compared to an untreated subject infected with the respiratory pathogen or an infected subject vaccinated with a non-mucosal vaccine. Antigen-specific IgAs and IgGs are increased in a subject infected with the respiratory pathogen subsequent to administration of the priming and boost compositions as compared to an untreated subject infected with the respiratory pathogen or an infected subject vaccinated with a non-mucosal vaccine.

Furthermore, histopathology scores are reduced in a subject infected with the respiratory pathogen subsequent to administration of the priming and boost compositions as compared to an untreated subject infected with the respiratory pathogen or an infected subject vaccinated with a non-mucosal vaccine or a formalin-inactivated vaccine that can induce pronounced pulmonary pathology. Histopathology scores may be based on a number of exemplary pathologic conditions, including but not limited to peribronchiolitis, perivasculitis, interstitial pneumonia and alveolitis.

### Definitions and Terminology

As used herein, the following terms shall have the following meanings:

The term “airborne pathogen” refers to any pathogen which is capable of being transmitted through the air and includes pathogens which travel through air by way of a carrier material and pathogens either artificially aerosolized or naturally occurring in the air.

As used herein, the phrase “respiratory pathogen infection” refers to infection transmitted by an airborne pathogen, such as a virus, bacterium, fungus or protozoa.

As used herein, the term “prophylactic” refers to the prevention of infection, the delay of infection, the inhibition of infection and/or the reduction of the risk of infection from pathogens, and includes pre- and post-exposure to pathogens. The prophylactic effect may, inter alia, involve a reduction in the ability of pathogens to enter the body, or may involve the removal of all or a portion of pathogens which reach airways and airway surfaces in the body from the body prior to the pathogens initiating or causing infection or disease. The airways from which pathogens may be removed, in whole or part, include all bodily airways and airway surfaces with mucosal surfaces, including airway surfaces in the lungs.

The term “immune response” refers to a response of a cell of the immune system, such as a B cell, T cell, dendritic cell, macrophage or polymorphonucleocyte (PMN), to a stimulus, such as an antigen or vaccine. An immune response can include any cell of the body involved in a host defense response, including for example, an epithelial cell that secretes an interferon or a cytokine. An immune response includes, but is not limited to, an innate and/or adaptive immune response. Methods of measuring immune responses are well known in the art and include, for example, measuring proliferation and/or activity of lymphocytes (such as B or T cells), measuring secretion of cytokines or chemokines, inflammation, antibody production and the like.

As used herein, “prophylactic immunity” and “prophylactic immune response” refer to condition of immunity or elicitation of an immune response against an infectious agent (e.g., prophylactically) that reduces, eliminates or decreases the duration of severity of an infection or at least one symptom of disease otherwise induced by the infectious agent.

The term “mucosal immune response” refers to an immune response in the mucosal tissues of a vertebrate subject. The mucosal immune response can comprise production of IgAs, particularly secretory IgAs, in mucosal tissue at a location in the vertebrate subject at mucosal administration site or a remote mucosal site, which is away from the site of mucosal administration of the antigen or antigen-adjuvant composition according to the present application.

The terms “protective immune response” and “protective immunity” refer to an immune response or state of immunity in which a subject's immune system can facilitate protection in a subject from an infection (e.g., prevents infection or prevents the development of disease associated with infection) or disease state or pathogen shedding characterized by the presence of one or more antigens ordinarily foreign to a host.

The terms “antigen” refers to a substance or molecule capable of eliciting an immune response and generating specific antibodies (humoral response) or cytotoxic T-lymphocytes (cell-mediated response) against it. As such, the antigen is capable of being recognized by components of the immune system, such as antibodies or lymphocytes. An antigen can be as small as a single epitope, or larger, and can include multiple epitopes. As such, the size of an antigen can be as small as about 5-12 amino acids (e.g., a peptide) and as large as: a partial protein, a full-length protein, including a multimer and fusion protein, chimeric protein, or agonist protein or peptide. In addition, antigens can include carbohydrates.

The term “immunogenic” refers to a reaction triggered by the presence of an epitope of an antigen or immunogen. The term “epitope” refers to an antigenic determinant that is sufficient to elicit an immune response. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein. Those of skill in the art will recognize that T cell epitopes are different in size and composition from B cell epitopes, and that epitopes presented through the Class I MHC pathway differ from epitopes presented through the Class II MHC pathway.

The term “vaccine” refers to a composition that induces an immune response in the recipient or host of the vaccine. A vaccine in accordance with the present disclosure is a composition comprising one or more protein antigen or immunogenic epitopes thereof in combination with one or more nucleic acid expression vectors encoding the one or more protein antigen or immunogenic epitopes thereof and one or more adjuvants. As such, a vaccine encompasses a DNA vaccine in combination with a protein (or subunit) vaccine and one or more adjuvants. The vaccine may be administered as a single formation or collectively in a series of formulations. The vaccine may induce a humoral (e.g., neutralizing antibody) response to one or more antigens, cell-mediated immune response (e.g., cytotoxic T lymphocyte (CTL)) response against one or more antigens, or both in a recipient so as to provide partial or complete protection against e.g., current or subsequent microbial infections or disease conditions characterized by the presence of e.g., one or antigens expressed in the recipient as a result of an infection.

The term “mucosal vaccine” refers to a vaccine or component thereof, which is directed against a respiratory pathogen and is administered to a subject by a mucosal route.

The term “non-mucosal vaccine” refers to a vaccine or component thereof, which is directed against a respiratory pathogen and is administered to a subject by a route other than a mucosal route.

The term “vaccination” refers to the administration of antigenic material to stimulate an individual's immune system to develop adaptive immunity to a pathogen or a host cell containing a non-natural antigen in a host. Vaccination can prevent or ameliorate of one or more symptoms associated with microbial infection or antigen- or epitope-specific cell associated with a disease, such as cancer; and/or lessening of the severity or frequency of one or more symptoms associated with the foregoing disease conditions.

The term “adjuvant” refers to a substance that enhances an immune response to an antigen, but is not antigenic itself when administered in the absence of an antigen. Adjuvants can enhance an immune response by several mechanisms including, e.g., lymphocyte recruitment, stimulation of B and/or T cells, stimulation of dendritic cells and/or stimulation of macrophages.

The term “immunize” is used with reference to providing a subject protection from an infectious disease or disease state, such as by vaccination.

The terms “protection”, “immune protection” and “protective response” are used interchangeably to convey partial or complete resistance to subsequent infections, active infections or certain disease conditions in a host. Neutralizing antibodies generated in a vaccinated host can provide this protection. In other situations, CTL responses can provide this protection. In some situations, both neutralizing antibodies and cell-mediated immune (e.g., CTL) responses provide this protection.

The term “control subject” refers to an unimmunized individual who is about the same age as the individual being vaccinated (to ensure that the effects of vaccination in the vaccinated individual and the control individual(s) are comparable). The subject can be a non-human animal, mammal or human. A human subject (also referred to as “patient” or “individual”) being vaccinated may be a fetus, infant, child, adolescent, or adult human. Non-human mammal subjects include, for example, domestic animals, laboratory animals, farm animals, captive wild animals and, most preferably, humans.

The term “polynucleotide” refers to deoxyribonucleotides or ribonucleotides and polymers thereof in either single- or double-stranded form. Unless specifically limited, the terms encompass nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues. The term “polynucleotide” or “polynucleotide sequence” can also be used interchangeably with gene, open reading frame (ORF), cDNA, mRNA encoded by a gene, and mRNA expressing a protein.

The terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein”, “polypeptide” and “peptide” are used interchangeably herein when referring to a gene product. Thus, exemplary polypeptides include gene products, naturally occurring proteins, homologs, orthologs, paralogs, fragments and other equivalents, variants, and analogs of the foregoing.

The term “variant” refers to protein or polypeptide that is different from the reference protein or polypeptide by one or more amino acids, e.g., one or more amino acid substitutions, but substantially maintains the biological function of the reference protein or polypeptide. The term “variant” further includes conservatively substituted variants. The term “conservatively substituted variant” refers to a peptide comprising an amino acid residue sequence that differs from a reference peptide by one or more conservative amino acid substitution, and maintains some or all of the activity of the reference peptide as described herein. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another, such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. The phrase “conservatively substituted variant” also includes peptides wherein a residue is replaced with a chemically derivatized residue, provided that the resulting peptide maintains some or all of the activity of the reference peptide as described herein. In some embodiments, the functional variant of a peptide shares a sequence identity of 70%, 75%, 80%, 85%, 90%, 91%, 92%, 93%, 94%, 95%, 96%, 97%, 98% or 99% with the reference peptide. For example, a functional variant of a protein may share a sequence identity of 70%, 75%, 80%, 85%, 90%, 91%, 92%, 93%, 94%, 95%, 96%, 97%, 98% and 99% with the reference version of the protein; and a functional variant of a fusion protein may shares a sequence identity of 70%, 75%, 80%, 85%, 90%, 91%, 92%, 93%, 94%, 95%, 96%, 97%, 98% and 99% with the reference fusion protein.

A variant of a polypeptide may be a fragment of the original polypeptide. The term “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 3, 5, 6, 8 or 10 amino acids long, at least 14 amino acids long, at least 20, 30, 40 or 50 amino acids long, at least 75 amino acids long, or at least 100, 150, 200, or more amino acids long.

The term “homologous amino acid sequence” used in this specification, unless otherwise stated herein, refers to an amino acid sequence derived from the substitution of one or more amino acids in the amino acid sequence of a polypeptide. Furthermore, the term “homologous polypeptide” used in this specification, unless otherwise stated herein, refers to a polypeptide homologue derived from the substitution of one or more amino acids in the amino acid sequence of a polypeptide.

The term “sequence identity,” as used herein, means that two peptide sequences are identical (i.e., on an amino acid-by-amino acid basis) over the window of comparison. The term “percentage of sequence identity” is calculated by comparing two optimally aligned sequences over the window of comparison, determining the number of positions at which the identical amino acid residue occurs in both sequences to yield the number of matched positions, dividing the number of matched positions by the total number of positions in the window of comparison (i.e., the window size), and multiplying the result by 100 to yield the percentage of sequence identity. The reference sequence may be a subset of a larger sequence, for example, as a segment of the full-length sequences of the compositions claimed in the present invention.

The term “expression cassette,” as used herein, refers to a DNA or RNA construct that contains one or more transcriptional regulatory elements operably linked to a nucleotide sequence coding the fusion protein of the present application. An expression cassette may additionally contain one or more elements positively affecting mRNA stability and/or an internal ribosome entry site (IRES) between adjacent protein coding regions to facilitate expression two or more proteins from a common mRNA.

The term “nucleic acid-based expression system,” as used herein, encompasses use of polynucleotide vectors, DNA or RNA vaccines, or other DNA or RNA based expression or delivery systems.

A nucleic acid sequence is “operably linked” to another nucleic acid sequence when the former is placed into a functional relationship with the latter. For example, a DNA for a presequence or signal peptide is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a signal peptide, contiguous and in reading phase. However, enhancers do not have to be contiguous. Linking is accomplished by ligation at convenient restriction sites. If such sites do not exist, synthetic oligonucleotide adaptors or linkers may be used in accordance with conventional practice.

The term “regulatory elements” refers to DNA/RNA sequences necessary for the expression of an operably linked coding sequence in one or more host organisms. The term “regulatory elements” is intended to include promoters, enhancers and other expression control elements (e.g., polyadenylation signals). Regulatory elements include those which direct constitutive expression of a nucleotide sequence in many types of host cells or those which direct expression of the nucleotide sequence only in certain host cells (e.g., tissue-specific regulatory elements). Expression cassettes generally contain sequences for transcriptional termination, and may additionally contain one or more elements positively affecting mRNA stability.

As used herein, the term “promoter” is to be taken in its broadest context and includes transcriptional regulatory elements (TREs) from genomic genes or chimeric TREs therefrom, including the TATA box or initiator element for accurate transcription initiation, with or without additional TREs (i.e., upstream activating sequences, transcription factor binding sites, enhancers, and silencers) which regulate activation or repression of genes operably linked thereto in response to developmental and/or external stimuli, and trans-acting regulatory proteins or nucleic acids. A promoter may contain a genomic fragment or it may contain a chimera of one or more TREs combined together.

The term “expression vectors,” as used herein, refers to recombinant expression vectors comprising nucleic acid molecules which encode the fusion proteins disclosed herein. Particularly useful vectors are contemplated to be those vectors comprising the expression cassette of the present application or those vectors in which the coding portion of the DNA segment is positioned under the control of a regulatory element. The expression vectors of the present application are capable of expressing the fusion protein of the present application in a cell transfected or infected by the expression vector. Expression vectors include non-viral vectors and viral vectors.

The term “non-viral vector,” as used herein, refers to an autonomously replicating, extrachromosomal circular DNA or RNA molecules, distinct from the normal genome. For example, a DNA plasmid or a RNA expressing cassette is a non-viral vector.

The terms “viral vector” and “recombinant virus” are used interchangeably herein to refer to any of the obligate intracellular parasites having no protein-synthesizing or energy-generating mechanism. The viral genome may be RNA or DNA contained with a coated structure of protein of a lipid membrane. The viruses useful in the practice of the present invention include recombinantly modified enveloped or non-enveloped DNA and RNA viruses, preferably selected from baculoviridiae, parvoviridiae, picornoviridiae, herpesviridiae, poxviridae, or adenoviridiae. The viral genomes may be modified by recombinant DNA techniques to include expression of exogenous transgenes and may be engineered to be replication deficient, conditionally replicating or replication competent. Chimeric viral vectors which exploit advantageous elements of each of the parent vector properties may also be useful in the practice of the present application. Minimal vector systems in which the viral backbone contains only the sequences need for packaging of the viral vector and may optionally include a transgene expression cassette may also be produced according to the practice of the present application. Although it is generally favored to employ a virus from the species to be treated, in some instances it may be advantageous to use vectors derived from different species which possess favorable pathogenic features. A viral vector may be derived from an adeno-associated virus (AAV), adenovirus, herpesvirus, vaccinia virus, poliovirus, poxvirus, a retrovirus (including a lentivirus, such as HIV-1 and HIV-2), Sindbis and other RNA viruses, alphavirus, astrovirus, coronavirus, orthomyxovirus, papovavirus, paramyxovirus, parvovirus, picornavirus, togaviruses and the like.

The term “retrovirus” refers to double-stranded RNA enveloped viruses that are primarily characterized by the ability to “reverse transcribe” their genome from RNA to DNA. The virions are 100-120 nm in diameter and contain a dimeric genome of the same plus RNA strand complexed with the nucleocapsid protein. The genome is encapsulated in a proteic capsid that also contains the enzymatic proteins required for viral infection, namely reverse transcriptase, integrase and protease. Matrix proteins form the outer layer of the capsid core that surrounds the viral nuclear particle and interacts with the envelope, a lipid bilayer derived from the host cell membrane. Immobilized in this bilayer is a viral envelope glycoprotein that is responsible for recognizing specific receptors on the host cell and initiating the infectious process.

The term “lentivirus” or “lentiviral vector” as used herein, refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “adeno-associated virus (AAV)” or “recombinant AAV (rAAV),” as used herein, refers to a group of replication-defective, nonenveloped viruses, that depend on the presence of a second virus, such as adenovirus or herpes virus or suitable helper functions, for replication in cells. AAV is not known to cause disease and induces a very mild immune response. AAV can infect both dividing and non-dividing cells and may incorporate its genome into that of the host cell. More than 30 naturally occurring serotypes of AAV are available. Many natural variants in the AAV capsid exist, allowing identification and use of AAV vectors with properties specifically suited for the cell targets of delivery. AAV vectors are relatively non-toxic, provide efficient gene transfer, and can be easily optimized for specific purposes. AAV viruses may be engineered using conventional molecular biology techniques to optimize the generation of recombinant AAV particles for cell specific delivery of the fusion proteins, for minimizing immunogenicity, enhancing stability, delivery to the nucleus, etc.

The terms “treat,” “treating” or “treatment” as used herein, refers to a method of alleviating or abrogating a disorder and/or its attendant symptoms. The terms “prevent”, “preventing” or “prevention,” as used herein, refer to a method of barring a subject from acquiring a disorder and/or its attendant symptoms. In certain embodiments, the terms “prevent,” “preventing” or “prevention” refer to a method of reducing the risk of acquiring a disorder and/or its attendant symptoms.

The term “inhibits” is a relative term, an agent inhibits a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an infection or a response, such as a pathological response, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

A “therapeutically effective amount,” as used herein, refers to an amount effective, at dosages and for periods of time necessary, to achieve the desired therapeutic result. A therapeutically effective amount of an expression vector may vary depending on the condition to be treated, the severity and course of the condition, the mode of administration, whether the agent is administered for preventive or therapeutic purposes, the bioavailability of the particular agent(s), the ability of the fusion protein or vector to elicit a desired response in the individual, previous therapy, the age, weight and sex of the patient, the patient's clinical history and response to the antibody, the type of the fusion protein or expression vector used, discretion of the attending physician, etc. A therapeutically effective amount is also one in which any toxic or detrimental effects of the expression vector is outweighed by the therapeutically beneficial effects. A “prophylactically or therapeutically effective amount” refers to an amount effective, at dosages and for periods of time necessary, to achieve the desired prophylactic or therapeutically result.

The terms, “improve”, “increase” or “reduce”, as used in this context, indicate values or parameters relative to a baseline measurement, such as a measurement in the same individual prior to initiation of the treatment described herein, or a measurement in a control individual (or multiple control individuals) in the absence of the treatment described herein.

As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions may comprise suitable solid or gel phase carriers or excipients. Exemplary carriers or excipients include but are not limited to, calcium carbonate, calcium phosphate, various sugars, starches, cellulose derivatives, gelatin, and polymers such as polyethylene glycols, liposomes and exosomes. Exemplary pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the therapeutic agents. As used herein, the term “pharmaceutically acceptable” refers to a molecular entity or composition that does not produce an adverse, allergic or other untoward reaction when administered to an animal or a human, as appropriate.

As used herein, the term “subject” includes both human and animal subjects. Thus, veterinary therapeutic uses are provided in accordance with the presently disclosed subject matter.

Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, fish, domesticated swine, ruminants, ungulates, horses (including race horses), poultry, and the like.

The term “mammal” refers to any animal classified as a mammal, including humans, non-human primates, domestic and farm animals, and zoo, sports, or pet animals, such as dogs, horses, cats, cows, etc. Preferably, the mammal is human.

As used herein, a “respiratory pathogen” refers to a microbial pathogen that is transmitted through air and gains entry into cells through the respiratory tract. The respiratory pathogen may be a virus, bacterium, fungus or protozoa. A respiratory pathogen of the present application includes a target protein antigen for use in the vaccine compositions of the present application.

As used herein, the terms “protein antigen” is intended to encompass all peptide or protein sequences which are capable of inducing an immune response within the animal concerned. The term “protein antigen” encompasses peptide or protein analogs of known or wild-type antigens, variant antigens that are more soluble or more stable than wild type antigen or that contain mutations or modifications rendering the antigen more immunologically active or optimized for expression in certain cell types (e.g., by codon optimization). An antigen may also be a peptide in which particular amino acid substitutions have been made to a naturally occurring antigen that alter protein structure, a portion of the naturally-occurring antigen including known protective epitopes (i.e. CTL epitopes), or a synthetically derived string of known epitopes that may or may not be limited to one pathogen (multivalent vaccine).

Further peptides or proteins that have sequences homologous with a desired antigen's amino acid sequence, where the homologous antigen induces an immune response to the respective pathogen, are also useful. Genes that are homologous to the desired antigen-encoding sequence should be construed to be included in the instant invention provided they encode a protein or polypeptide having a biological activity substantially similar to that of the targeted antigen.

Variants or analogs of the antigens described herein can differ from naturally occurring proteins or peptides by conservative amino acid, sequence differences or through modifications that do not affect sequence, or by both. For example, conservative amino acid changes may be made, which although they alter the primary sequence of the protein or peptide, do not normally alter its function. Modifications (which do not normally alter primary sequence) include in vivo, or in vitro chemical derivatization of polypeptides, e.g., acetylation, or carboxylation. Also included as antigens are proteins modified by glycosylation, e.g., those made by modifying the glycosylation patterns of a polypeptide during its synthesis and processing or in further processing steps, e.g., by exposing the polypeptide to enzymes which affect glycosylation, e.g., mammalian glycosylating or deglycosylating enzymes. Also included as antigens according to this invention are proteins having phosphorylated amino acid residues, e.g., phosphotyrosine, phosphoserine, or phosphothreonine. Also included as antigens are polypeptides that have been modified using ordinary molecular biological techniques, so as to improve their resistance to proteolytic degradation or to optimize solubility properties. Analogs of such polypeptides include those containing residues other than naturally occurring L-amino acids, e.g., D-amino acids or non-naturally occurring synthetic amino acids. The antigens of the invention are not limited to products of any of the specific exemplary processes listed herein.

An antigen can be a full-length or a truncated antigen, an immunogenic fragment thereof, or an epitope derived from the antigen. In certain embodiments, the pathogen-specific antigen in the boosting compositions may be in the form of an attenuated or killed pathogen. Effective antigens also include surface antigens of these pathogens.

The term “epitope” as used herein refers to a sequence of at least about 3 to 5, preferably about 5 to 10 or 15, and not more than about 1,000 amino acids (or any integer therebetween), which define a sequence that by itself or as part of a larger sequence, binds to an antibody generated in response to such sequence or stimulates a cellular immune response. The term “epitope” encompasses sequences identical to the native sequence, as well as modifications to the native sequence, such as deletions, additions and substitutions (generally conservative in nature). The antigens used in the invention may comprise only a single epitope, such as, for example, a single CTL epitope.

### Compositions for Inducing a Mucosal Immune Response Against a Respiratory Pathogen

The methods described herein can be applied to a variety of respiratory pathogens. In the mucosal vaccine compositions of the present application, respiratory pathogen proteins and polynucleotide vectors encoding the same are derived from respiratory pathogens. An aspect of the present application relates to a pharmaceutical composition comprising the priming and boost composition described herein. In addition to the antigen-encoded polynucleotide vectors, the antigenic protein and adjuvants described herein, a pharmaceutical composition of the present application will include no one or more pharmaceutically acceptable carriers.

The antigens encoded by the nucleic acids in the priming composition or boosting compositions and the protein antigens in the boosting compositions preferably have overlapping epitopes. In certain embodiments, the two antigens may be identical to each other. Alternatively, the two antigens may have overlapping but different set of epitopes. By way of an illustrating example, in a vaccination protocol for RSV, a DNA encoding an RSV full-length glycoprotein may be used in the priming composition, and the boosting composition may be an ectodomain of glycoprotein. By way of another illustrating example, the priming composition may be a vector encoding an RSV antigen, and the boosting composition may comprise a protein form of the full-length or a portion of antigen, or vice versa.

In certain preferred embodiment, the respiratory pathogen is a virus. Exemplary respiratory viruses for use in accordance with the present application include, but are not limited to pneumoviruses, such as respiratory syncytial virus (RSV); human coronaviruses, such as severe acute respiratory syndrome coronavirus Type 2 (SARS-CoV-2), SARS-CoV-1, MERS-CoV, OC43, 229E, NL (NH), and HKUI; non-human coronaviruses, such as infectious bronchitis virus (poultry), porcine transmissible gastroenteric virus (pig), porcine hemagglutinating encephalomyelitis virus (pig), feline infectious peritonitis virus (cat), feline enteric coronavirus (cat), and canine coronavirus (dog); influenza viruses, including Type A, Type B, and Type C influenza viruses, including various subtypes or serotypes thereof, parainfluenza viruses, such as human parainfluenza virus (HPIV), parainfluenza Virus Type 1, parainfluenza Virus Type 3, bovine parainfluenza Virus Type 3, rubulavirus (mumps virus, parainfluenza Virus Type 2, parainfluenza virus Type 4; paramyxoviruses, including Newcastle disease virus (chickens), rinderpest, morbilliviruses, such as Measles morbillivirus, which is known to cause measles, and canine distemper virus (CDV), which is known to cause canine distemper; metapneumoviruses, such as human metapneumovirus (HIMNV); picornaviruses, including echoviruses and rhinoviruses, such as human rhinovirus (HRV), which are known to cause the common cold; respiratory adenoviruses; bocaviruses, such as human bocavirus (HBoV); and varicella zoster virus (VZV), which is known to cause chickenpox.

Other airborne respiratory viruses include, arenaviruses (including funin, machupo, and Lassa), filoviruses (including Marburg and Ebola), hantaviruses; paramyxovirus, morbillivirus, togavirus, coxsackievirus, parvovirus Bl 9, reoviruses, variola (Variola major (Smallpox)), monkey poxviruses and poxviruses, including e.g., vaccinia viruses that cause Cowpox.

Viral hemorrhagic fevers are caused by members of the arenavirus family (Lassa fever) (which family is also associated with Lymphocytic choriomeningitis virus (LCMV)), filovirus (Ebola virus), and hantavirus (puremala). The parvovirus family includes feline parvovirus (feline enteritis), feline panleucopeniavirus, canine parvovirus, and porcine parvovirus. The adenovirus family includes viruses (e.g.,) which are known to cause respiratory diseases.

In certain preferred embodiments, the respiratory pathogen is a virus selected from the group consisting of respiratory syncytial virus (RSV), SARS-CoV-2, SARS-CoV-1, MERS-CoV, a Type A influenza virus, a human rhinovirus, or varicella zoster virus. In certain preferred embodiments, the virus is RSV or SARS-CoV-2 plus emerging mutants or influenza viruses.

In a preferred embodiment, the respiratory pathogen is RSV. In more particular embodiments, the antigen is an RSV antigen selected from the group consisting of fusion (F) protein, prefusion-F (pre-F) protein, glycoprotein G, small hydrophobic (SH), protein phosphoprotein (P), nucleoprotein (N) protein, matrix (M) protein, large (L) protein, M2-4 regulatory protein, M2-2 regulatory protein, non-structural protein NS1, and the non-structural protein NS2. In a preferred embodiment, the RSV antigen is a full-length F protein or prefusion-F (pre-F) protein. In other embodiments, the RSV antigen is a processed form of F, a truncated F protein, an extracellular domain of pre-F, an immunogenic fragment thereof, or a secreted form thereof. In some embodiments, the forms of F protein described herein may include signal peptides and/or various purification tags known in the art (e.g., histidine tags etc.).

Variants and mutants of full-length or truncated forms of F protein or pre-F protein can also be used and are described in U.S. Pat. Nos. 9,675,685, 9,950,058, 10,017,543, and 10,022,437, the disclosures of which are incorporated by reference in their entirety herein.

In another embodiment, the respiratory pathogen is SARS-CoV-2 or mutant. In particular embodiments, the antigen target for vaccination is a SARS-CoV-2 antigen may include the spike (S) protein, envelope (E) protein, membrane (M) protein, nucleocapsid (N) protein, orf1a, orf1b, orf3a, E, M, orf6, orf7a, orf8, M, orf10, as well as immunogenic fragments thereof and consensus proteins thereof, which are derived from polynucleotide or protein sequences from any variant SARS-CoV-2 isolate. In a preferred embodiment, the SARS-CoV-2 or mutant is the spike (S) protein or a portion of the S protein.

In another specific embodiment, the respiratory pathogen is a Type I influenza virus or combination thereof. Influenza viruses from the orthomyxoviridae family include: Type A influenza subtypes, Type B influenza subtypes, and Type C influenza subtypes. Type A influenza viruses are the most virulent human pathogens. Influenza Type A viruses are divided into subtypes on the basis of two proteins on the surface of the virus: hemagglutinin (HA) and neuraminidase (NA). There are 18 known HA subtypes and 11 known NA subtypes. Many different combinations of HA and NA proteins are possible. For example, an “H7N2 virus” designates an influenza A virus subtype that has an HA 7 protein and an NA 2 protein.

Similarly, an “H5N1” virus has an HA 5 protein and an NA 1 protein. Type A influenza viruses include a variety of sub-types or serotypes, including those associated with pandemics including, HINI, which caused Spanish Flu in 1918, and Swine Flu in 2009; H2N2, which caused Asian Flu in 1957; H3N2, which caused Hong Kong Flu in 1968; and H5N1, which caused Bird Flu in 2004.

In certain particular embodiments, the Type A influenza virus is a subtype selected from the group consisting of H1N1, H3N2, H5N1, H5N2, H5N3, H5N4, H5N5, H5N6, H5N7, H5N8, and H5N9, H7N1, H7N2, H7N3, H7N4, H7N5, H7N6, H7N7, H7N8, and H7N9, H9N1, H9N2, H9N3, H9N4, H9N5, H9N6, H9N7, H9N8, H9N9, H1 7N10, H18N1 1, or combination thereof. In certain particular embodiments, the priming composition comprises a Type A influenza-encoded polynucleotide, where each polynucleotide of the first boost, second boost, or both, comprises a Type A influenza antigen hemagglutinin antigen (HA), a neuraminidase (NA) antigen, or both.

In some embodiments, the respiratory pathogen is a bacterium. Exemplary airborne bacteria known to cause disease, include, but are not limited to Streptococcus spp., such as S. pneumoniae (pneumonia)(including 23 serotypes thereof), S. pyogenes (scarlet fever), S. oralis, and S. mitis; Haemophilus spp., such as H. influenzae (flu)(e.g., types a, b, c, d, e, f), H. parainfluenzae, and H. somnus; Mycobacterium spp., such as M tuberculosis (tuberculosis (TB)), M kansasii (TB), and M avium (pneumonia); Staphylococcus aureus (pneumonia); Bordetella pertussis (whooping cough); Bacillus anthracis (anthrax); Chlamydia spp., such as C. psittaci (pneumonia) and C. pneumoniae (pneumonia); Neisseria spp., such as N meningitides(meningitis); Klebsiella pneumonia (pneumonia); Pseudomonas spp., such as P. aeruginosa (pneumonia), P. pseudomallei (pneumonia), and P. mallei (pneumonia); Acinetobacter spp. (pneumonia), Mycoplasma spp., such as M pneumoniae (pneumonia); Brucella spp. (brucellosis), such as B. suis, B. melitensis, B. abortus and B. canis, Francisella tularensis (pneumonia/fever), Legionella pneumonia (Legionnaires disease), Nocardia asteroides (pneumonia), Corynebacteria diphtheria (diphtheria); Actinobacillus actinomycetemcomitans; Moraxella spp., such as M catarrhalis and M lacunata; Alkaligenes spp., Cardiobacterium spp.; Fusobacterium nucleatum; Actinomyces; M fermentans and M pneumonia; Burkholderia spp., such as B. pseudomallei; Coxiella burnetii (Q fever); and Rickettsia spp., such as R. prowazekii, R. rickettsii, R. conorrii and R. typhi.

In certain particular embodiments, the bacterium is Streptococcus pneumonia, Staphylococcus aureus, Haemophilus influenzae, Mycobacterium tuberculosis, Bordetella pertussis or Bacillus anthracis.

Bacterial antigens that may be targeted for vaccination with the compositions of the present application may include any bacterial antigens from the above-described bacteria. Specific antigens for M. tuberculosis include e.g., Rv2557, Rv2558, RPFs: Rv0837c, Rvl884c, Rv2389c, Rv2450, Rv1009, aceA (Rv0467), PstSl, (Rv0932), SodA (Rv3846), Rv203 1c 16 kDal., Tb Ra12, Tb H9, Tb Ra35, Tb38-1, Erd 14, DPV, MTI, MSL, mTTC2 and hTCCl (WO99/51748). M. tuberculosis antigens also include fusion proteins and variants thereof where at least two, preferably three polypeptides of M. tuberculosis are fused into a larger protein.

Preferred fusions include Ral2-TbH9-Ra35, Erd 14-DPV-MTI, DPV-MTI-MSL, Erd 14-DPV-MTI-MSL-mTCC2, Erd14-DPV-MTI-MSL, DPV-MTI-MSL-mTCC2, TbH9-DPV-MTI (WO 99/51748).

Specific antigens derived from Streptococcus spp, include those derived from S. pneumoniae (e.g., PsaA, PspA, streptolysin, choline-binding proteins), the protein antigen pneumolysin, and mutant detoxified derivatives thereof (WO 90/06951; WO 99/03884).

Specific antigens derived from Haemophilus spp., include e.g., H. influenzae antigens PRP, OMP26, high molecular weight adhesins, PS, P6, protein D and lipoprotein D, fimbrin and fimbrin derived peptides (U.S. Pat. No. 5,843,464) or multiple copy variants or fusion proteins thereof.

Specific Chlamydia antigens for use include, e.g., the High Molecular Weight Protein (HWMP) (WO 99/17741), ORF3 (EP 366 412), and putative membrane proteins (Pmps). Other Chlamydia antigens for use are described in WO 99/28475.

In some embodiments, the respiratory pathogen is a fungus. Exemplary infectious diseases may be caused by airborne fungi including, e.g., Aspergillus species, Absidia corymbifera, Rhixpus stolonifer, Mucor plumbeaus, Cryptococcus neoformans, Histoplasm capsulatum, Blastomyces dermatitides, Coccidioides immitis, Penicillium species, Micropolyspora faeni, Thermoactinomyces vulgaris, Alternaria alternate, Cladosporium species, Helminthosporium, and Stachybotrys species. Other respiratory pathogens and their antigens are described in U.S. Patent Application Publication No. 2019/0216841.

### Adjuvants

The development of therapeutic vaccines for viruses and other pathogens has focused on the activation of CTL and/or NAbs to recognize and destroy infected cells and/or controlling the virus spread. The methods of the invention are effective in enhancing cellular immune responses, making them suitable for providing therapeutic vaccination. The effectiveness of the disclosed methods is preferably enhanced by inclusion of cytokine adjuvants and CpG motifs.

Accordingly, the priming or boost compositions will generally include one or more adjuvant(s) to further boost an immune response. The adjuvants may be administering before, at the same time as, or after administration of the vaccine composition. An adjuvant is a substance or procedure which augments specific immune responses to antigens by modulating the activity of immune cells.

The development of newer, safer adjuvants has recently focused on stimulating particular immune response pathways. In some embodiments, coadministration of cytokines, such as interferon-y and granulocyte-macrophage colony stimulating factor (GM-CSF) may be included to stimulate cellular immune responses (reviewed in (Petrovsky and Aguilar, Immunol. Cell Biol. 82:488-496 (2004)). High levels of cytokines can cause toxicity, however, which can necessitate the careful testing and monitoring of multiple dosing regimens. Administration of cytokines or cytokine-encoding expression vectors may be included with the polynucleotide vectors in the priming composition or in expression vectors co-expressing genes encoding the respiratory pathogen protein and the cytokine for simultaneous expression by the same vector.

Exemplary adjuvants include, but are not limited to, CpG oligodeoxynucleotides (ODNs), adjuvants comprising monophosphoryl lipid A (MPL) or derivatives therefrom, such as 3 De-O-acylated monophosphoryl lipid A, 3-O-desacyl-4′-monophosphoryl lipid A, AS01, AS02 and AS04; water-in-oil or oil-in-water emulsions (e.g. Freund's adjuvant (complete and incomplete), MONTANIDE™ ISA 51, MONTANIDE™ ISA 720 VG MONTANIDE™ ISA 50V, MONTANIDE™ ISA 206, MONTANIDE™ IMS 1312, MF59® and AS03); bacterial-derived adjuvants, such as lipopolysaccharides (LPS) and bacterial toxins; adjuvant emulsions enabling the slow release of antigen; agonistic antibodies to co-stimulatory molecules; muramyl dipeptides, recombinant/synthetic adjuvants, alum-based salts, aluminum salts (e.g. aluminum hydroxide, aluminum phosphate and potassium aluminum sulfate (also referred to as Alum)), liposomes, virosomes, exosomes, saponin-based adjuvants, including saponin-based adjuvants (e.g., Iscoms, Iscom matrices, ISCOMATRIX™ adjuvant, MATRIX-M™ adjuvant, MATRIX-C™ adjuvant, Matrix Q™ adjuvant, ABISCO™-100 adjuvant, and ABISCO™-300 adjuvant; ISCOPREP™ adjuvants and derivatives, including QS-21 and QS-21 derivatives; saponin derivatives from, e.g., Quillaja saponaria, Panax ginseng, Panax notoginseng, Panax quinquefolium, Platycodon grandiflorum, Polygala senega, Polygala tenuifolia, Quillaja brasiliensis, Astragalus membranaceus and Achyranthes bidentate; polyamino acids, co-polymers of amino acids, paraffin oil, Regressin (Vetrepharm, Athens GA), Avridine, liposomes, oil, Corynebacterium parvum, Bacillus Calmette Guerin, iron oxide, sodium alginate, glucan, MF59 (Novartis), and dextran sulfate. Additionally, CTL responses can be primed by conjugating a protein antigen to lipids, such as tripalmitoyl-S-glycerylcysteinyl-seryl-serine (P3CSS). In certain embodiments, the vaccine composition includes QS-21 at 50 μg/dose/subject.

In certain particular embodiments, the adjuvant is a toll-like receptor (TLR) agonist, such as TLR-9. Exemplary TLR-9 adjuvants include (among others) those containing CpG DNA motifs commonly found in bacterial DNA CpG oligonucleotides (ODNs) are potent activators of cellular immune responses. In particular, CpG ODNs can improve the function of professional antigen-presenting cells and boost the generation of humoral and cellular vaccine-specific immune responses. These effects can be optimized by maintaining ODNs and vaccine in close proximity. Consequently, CpG ODNs may be included in the vaccine formulations of the present application. Exemplary CpG ODNs include CpG 7909, CpG 1080, ODN 2216, ODN 21798, ODN 2007, ODN D-SLO1, MGN 1703, K3-SPG, DYNAVAX 1018, and combinations thereof. In certain preferred embodiments, the compositions of the present application may include CpG 7909 alone or CpG 7909 in combination with monophosphoryl lipid A (MPL).

In other embodiments, the adjuvant is a TLR-4 ligand, such as monophosphoryl lipid A (MPL), or TLR-7 ligand, such as R837. TLR-4 and TLR-7 ligands in combination with a nanoparticle formulation have been reported can enhance and prolong antibody responses when administered with antigen following immunization (Kasturi et al. (2011) Nature, Vol. 470: 543-560).

In some embodiments, it may be advantageous to employ surfactants in the pharmaceutical composition of the present application, where those surfactants will not be disruptive to the pharmaceutical composition which is administered. Surfactants or anti-adsorbents that prove useful include polyoxyethylenesorbitans, polyoxyethylenesorbitan monolaurate, polysorbate-20, such as Tween-20™, polysorbate-80, polysorbate-20, hydroxycellulose, genapol and BRIJ surfactants. By way of example, when any surfactant is employed in the present disclosure to produce a parenterally administrable composition, it is advantageous to use it in a concentration of about 0.01 to about 0.5 mg/ml.

### Antigen-Encoding Polynucleotide Expression Vectors

The polynucleotide expression vector used in the priming composition or boosting compositions may be RNA such as mRNA, or DNA such as genomic DNA, synthetic DNA or cDNA. In order to obtain expression of the antigenic peptide within mammalian cells, it is necessary for the nucleotide sequence encoding the antigenic peptide to be presented in an appropriate vector system. By “appropriate vector” is meant any DNA vector or any mRNA formulations in various delivery vehicles that will enable the antigenic peptide to be expressed within a mammal in sufficient quantities to evoke an immune response.

For example, the polynucleotide expression vector selected may be a plasmid, a phagemid or a viral vector. Typically, the vector includes promoter/enhancer sequences and polyadenylation/transcriptional termination sequences appropriately arranged to provide expression of the antigenic proteins described herein. The construction and use of polynucleotide expression vectors including these and other components are well known to those skilled in the art.

Generally, the polynucleotide expression vector in the priming or boosting composition is under the control of a suitable promoter for efficient expression. As used herein, “under the control of” or “operably linked” means that the promoter is in the correct location and orientation in relation to a polynucleotide encoding the antigen to control the initiation of transcription by RNA polymerase and expression of the polynucleotide. Suitable promoter/enhancer elements for use in the polynucleotide expression vector include, but are not limited to, human cytomegalovirus (CMV) immediate early gene promoter, the SV40 early promoter, the Rous sarcoma virus long terminal repeat, RNA pol I, pol II, and pol III promoters. In certain embodiments, the promoter is a CMV promoter, preferably a CMV immediate early gene promoter.

A priming composition containing a polynucleotide expression vector encoding a respiratory pathogen antigen for vaccination can be administered in a variety of ways. The polynucleotide expression vector can be administered in a naked form (alone), incorporated into a viral vector, encapsulated in a liposome, or combined with one or more transfection facilitating agents or microneedles. The polynucleotide expression vector may be suspended in an appropriate medium, for example a buffered saline solution, such as PBS, and then injected intramuscularly, subcutaneously, intradermally or mucosally or administered using a gene gun or other electronic (e.g., electroporation) devices, microneedles and the like. In certain preferred embodiments, the polynucleotide expression vector encoding a respiratory pathogen antigen, such as RSV prefusion F (pre-F) protein, is administered in a naked form via intramuscular injection.

In another embodiment, the polynucleotide expression vector is administered by intradermal administration, preferably via use of gene-gun (particularly particle bombardment) administration techniques. Such techniques may involve coating of the vector on to gold beads which are then administered under high pressure into the epidermis, such as, for example, as described in Haynes et al. J. Biotechnology 44: 37-42 (1996).

In some embodiments, a viral vector may be used to deliver the polynucleotide expression vector. Such an approach can provide for abundant expression of DNA-encoded proteins in multiple cell types, strong enhancement of CTL and antibody responses and the ability of the virus to encode multiple genes. Exemplary virus vectors for delivery and expression of the protein antigens include replication-defective adenoviruses, adeno-associated viruses, lentiviruses and vaccinia viruses.

In yet other embodiments, the vectors can be encapsulated by, for example, in liposomes or within polylactide co-glycolide (PLG) particles for administration via the nasal or pulmonary routes.

In certain embodiments, the polynucleotide vectors of the present application may be further modified to promote increased expression, ensure proper folding, provide a GC content suitable for increasing mRNA stability or reducing secondary structures, minimize tandem repeat codons or base runs that may impair gene construction or expression, insert or remove protein trafficking sequences, remove/add post translation modification sites in an encoded protein (e.g. glycosylation sites), add, remove or shuffle protein domains, insert or delete restriction sites, modify ribosome binding sites and mRNA degradation sites, adjust translational rates to allow the various domains of the protein to fold properly, or reduce or eliminate problem secondary structures within a polynucleotide.

In certain preferred embodiments, the polynucleotide sequence in the polynucleotide expression vector encoding a respiratory pathogen protein antigen is codon-optimized for expression in human and non-human mammals. Such polynucleotide sequences can be codon-optimized using optimization algorithms known in the art. For example, in certain embodiments, the antigen-encoding sequences are codon optimized for expression in humans, wherein the codon sequences are replaced with e.g., “humanized” codons (e.g., codons that appear frequently in highly expressed human genes).

In some embodiments, the codon optimized polynucleotide sequence shares less than 95%, less than 90%, less than 85%, less than 80%, less than 75%, less than 70%, less than 65%, less than 60%, less than 55% or less than 50% sequence identity to a naturally-occurring or wild-type sequence (e.g., a naturally-occurring or wild-type DNA or mRNA sequence encoding a respiratory viral infection polypeptide of interest (e.g., an antigenic protein or polypeptide).

In some embodiments, the codon optimized polynucleotide sequence shares between 50% and 95%, between 50% and 90%, between 50% and 85%, between 50% and 80%, between 50% and 75%, between 50% and 70%, between 50% and 65%, between 50% and 60%, between 50% and 55%, between 55% and 95%, between 55% and 90%, between 55% and 85%, between 55% and 80%, between 55% and 75%, between 55% and 70%, between 55% and 65%, between 55% and 60%, between 60% and 95%, between 60% and 90%, between 60% and 85%, between 60% and 80%, between 60% and 75%, between 60% and 70%, between 60% and 65%, between 65% and 95%, between 65% and 90%, between 65% and 85%, between 65% and 80%, between 65% and 75%, between 65% and 70%, between 70% and 95%, between 70% and 90%, between 70% and 85%, between 70% and 80%, between 70% and 75%, between 75% and 95%, between 75% and 90%, between 75% and 85%, between 75% and 80%, between 80% and 95%, between 80% and 90%, between 80% and 85%, between 85% and 95%, between 85% and 90%, or between 90% and 95% sequence identity to a naturally-occurring or wild-type sequence encoding a respiratory pathogen protein of interest (i.e., antigenic protein).

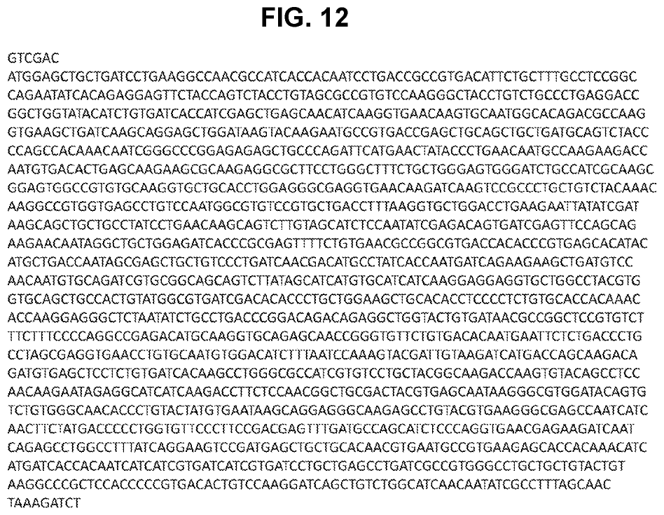

In a preferred embodiment, the polynucleotide vector encodes a codon-optimized pre-F comprising the nucleotide sequence shown in FIG. 12.

In some embodiments, the polynucleotide vector for expressing a respiratory pathogen protein antigen is optimized to include, eliminate, increase or decrease cis-acting motifs, such as internal TATA-boxes, chi-sites, ribosomal entry sites, AT-rich or GC-rich sequence stretches, ARE, INS, CRS sequence elements, cryptic splice donor and acceptor sites, and/or branch points.

In other embodiments, two or more antigens may be expressed in the polynucleotide vectors of the present application. For example, in the priming boosting compositions having an antigen-encoding polynucleotide vector, the two or more antigens may be fusion proteins in which either the full-length antigenic proteins or immunogenic fragments thereof are expressed from a single open-reading frame (e.g. expressed as a single transcript). In other embodiments, the two or more antigens may be expressed from different open-reading frames (e.g. expressed as separate transcripts) under the control of a single promoter or different promoters with or without internal ribosome entry sites (IRES) known to those of skill in the art. Corresponding, in the boosting compositions, the two or more protein antigens may be present as a mixture of antigens or as one or more fusion proteins. The two or more antigens may be from a single pathogen or multiple pathogens.

### Expression Cassette

Another aspect of the application relates to an expression cassette that comprises one or more regulatory sequences operably linked to the coding sequence of the protein antigen of the present application.

In some embodiments, the one or more regulatory sequences include a promoter and a '3 UTR sequence. Preferred promoters are those capable of directing high-level expression in a target cell of interest. The promoters may include constitutive promoters (e.g., HCMV, SV40, elongation factor-1α (EF-1α)) or those exhibiting preferential expression in a particular cell type of interest. In some embodiments, a ubiquitous promoter such as a CMV promoter or a CMV-chicken beta-actin hybrid (CAG) promoter to control the expression of the fusion protein of the present application. In other embodiments, a tissue specific promoter, such as skin specific promotor, neuron specific promotor, muscle specific promoter and liver specific promoter, is used to control the expression of the fusion protein in a specific tissue. Tissue specific promoters are well known in the art.

In some embodiments, it is contemplated that certain advantages will be gained by positioning the coding sequence under the control of a recombinant, or heterologous, promoter. As used herein, a recombinant or heterologous promoter is intended to refer to a promoter that is not normally associated with a protein's gene in its natural environment. Such promoters may include promoters isolated from plant, insect, bacterial, viral, eukaryotic, fish, avian or mammalian cells. Naturally, it will be important to employ a promoter that effectively directs the expression of the DNA segment in the cell type chosen for expression. The use of promoter and cell type combinations for protein expression is generally known to those of skill in the art of molecular biology.

In some embodiments, the one or more regulatory sequences further comprise an enhancer. Enhancers generally refer to DNA sequences that function away from the transcription start site and can be either 5′ or 3′ to the transcription unit. Furthermore, enhancers can be within an intron as well as within the coding sequence. They are usually between 10 and 300 bp in length, and they function in cis. Enhancers function to increase and/or regulate transcription from nearby promoters. Preferred enhancers are those directing high-level expression in the antibody producing cell.

In some embodiments, cell or tissue-specific transcriptional regulatory elements (TREs) can be incorporated into expression cassette to restrict expression to desired cell types. An expression vector may be designed to facilitate expression of the fusion proteins herein in one or more cell types.

### Non-Viral Vectors

In some embodiments, the expression vector is a non-viral expression vector. In some embodiments, the non-viral expression vector is a plasmid capable of expressing the fusion protein of the present application in an in vitro and/or in vivo setting.

In some embodiments, non-viral expression vectors of the present application are introduced into cells or tissues by encapsulating the expression vectors in liposomes, microparticles, microcapsules, virus-like particles, or erythrocyte ghosts, or exosomes. Such compositions can be further linked by chemical conjugation to, for example, microbial translocation domains and/or targeting domains to facilitate targeted delivery and/or entry of nucleic acids into the nucleus of desired cells to promote gene expression. In addition, plasmid vectors may be incubated with synthetic gene transfer molecules such as polymeric DNA-binding cations like polylysine, protamine, and albumin, and linked to cell targeting ligands such as asialoorosomucoid, insulin, galactose, lactose or transferrin.

In some embodiments, non-viral expression vectors are introduced into the cells or tissues as naked DNA by direct injection or electroporation, or as mRNA vaccine formulations. Uptake efficiency of naked DNA may be improved by compaction or by using biodegradable latex beads. Such delivery may be improved further by treating the beads to increase hydrophobicity and thereby facilitate disruption of the endosome and release of the DNA into the cytoplasm.

### Viral Vectors

In some embodiments, the expression vector of the present application is a viral expression vector. In certain embodiments, viral expression vectors may be engineered to target certain diseases and cell populations by using the targeting characteristics inherent to the virus vector or engineered into the virus vector. Specific cells may be “targeted” for delivery of polynucleotides, as well as expression. Viral vectors may be preferable in acting as the prime in the methods discussed herein.

In some embodiments, the viral expression vector is selected from the group consisting of retroviral vectors, lentivirus vectors, adenovirus vectors, adeno-associated virus (AAV) vectors and herpes virus vectors.

In some embodiments, the viral expression vector is a lentivirus vector. In some embodiments, the lentivirus vector is a non-primate lentivirus vector, such as equine infectious anemia virus (EIAV).

In some embodiments, the viral expression vector comprises a mitogenic T cell-activating transmembrane protein and/or a cytokine-based T cell-activating transmembrane protein in the viral envelope. In some embodiments, the viral expression vector is a lentiviral vector comprising a mitogenic T cell-activating transmembrane protein and/or a cytokine-based T cell-activating transmembrane protein in the viral envelope.

In some embodiments, the viral expression vector is a recombinant AAV vector (rAAV). rAAVs can spread throughout CNS tissue following direct administration into the cerebrospinal fluid (CSF), e.g., via intrathecal and/or intracerebral injection. In some embodiments, rAAVs (such as AAV-9 and AAV-10) cross the blood-brain-barrier and achieve wide-spread distribution throughout CNS tissue of a subject following intravenous administration. In some cases, intravascular (e.g., intravenous) administration facilitates the use of larger volumes than other forms of administration (e.g., intrathecal, intracerebral). Thus, large doses of rAAVs (e.g., up to 1015 rAAV genome copies (GC)/subject) can be delivered at one time by intravascular (e.g., intravenous) administration. Methods for intravascular administration are well known in the art and include, for example, use of a hypodermic needle, peripheral cannula, central venous line, etc.

Any suitable AAV serotype may be utilized for the recombinant AAV, including but not limited to AAV1, AAV2, AAV3, AAV4, AAV5, AAV6, AAV7, AAV8, AAV9, AAV10, AAV 11, AAV12, and pseudotyped combinations thereof. Pseudotyped (or chimeric) AAV vectors include portions from more than one serotype, for example, a portion of the capsid from one AAV serotype may be fused to a second portion of a different AAV serotype capsid, resulting in a vector encoding a pseudotyped AAV2/AAV5 capsid. Alternatively, the pseudotyped AAV vector may contain a capsid from one AAV serotype in the background structure of another AAV serotype. For example, a pseudotyped AAV vector may include a capsid from one serotype and inverted terminal repeats (ITRs) from another AAV serotype. Exemplary AAV vectors include recombinant pseudotyped AAV2/1, AAV2/2, AAV2/5, AAV2/7, AAV2/8 and AAV2/9 serotype vectors. Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV11, AAV12 or other known or as yet unknown AAV serotypes. These ITRs or other AAV components may be readily isolated from an AAV serotype using techniques available to those of skill in the art. In addition, AAV sequences may be isolated or obtained from academic, commercial, or public sources (e.g., the American Type Culture Collection, Manassas, Va.) or may be obtained through synthetic or other suitable means by reference to published sequences such as are available in the literature or in databases such as, e.g., GenBank, PubMed and the like.

It will be appreciated by those skilled in the art that the design of the expression vector of the present application can depend on such factors as the choice of the host cell to be transformed, the level of expression of protein desired, and the like.

### Methods for Inducing a Mucosal Immune Response Against a Respiratory Pathogen

One aspect of the application is a method for treating or preventing symptoms of a respiratory pathogen infection in a subject. The method comprises the steps of (1) administering to the subject an effective amount of a priming composition comprising a polynucleotide vector encoding an antigenic peptide (or protein antigen) of the respiratory pathogen, and (2) administering to the subject an effective amount of a boosting composition comprising the antigenic peptide (or protein antigen) of the respiratory pathogen, wherein the priming composition is administered subcutaneously or intramuscularly, and the boosting composition is administered through a mucosal route.

In some embodiments, the priming composition is administered by a non-mucosal route (e.g., intramuscularly, intravenously, intraperitoneally, intradermally, or subcutaneously) and the boosting composition (which is also referred to as the “mucosal vaccine”) is administered intranasally or by inhalation. In some embodiments, the boosting composition is administered multiple times with an interval of 1 to 26 weeks between doses. In some embodiments, the boosting composition further comprises one or more adjuvants. In some embodiments, the priming and/or boosting composition are administered prior to the subject's exposure to the respiratory pathogen. In some embodiments, the priming and/or boosting composition are administered after the subject's exposure to the respiratory pathogen.

Another aspect of the application is a method for preventing or ameliorating symptoms of a respiratory syncytial virus (RSV) infection in a subject comprising prophylactically or therapeutically administering to the mammal: a priming composition comprising a polynucleotide expression vector encoding RSV antigen under the control of a promoter; a first boost comprising one or more aerosolized formulations comprising the RSV antigen alone or in combination with the polynucleotide expression vector; and one or more adjuvants, where the polynucleotide expression vector in the priming composition encodes the same RSV antigen present in the first boost, where the one or more aerosolized formulations in the first boost comprise a CpG oligonucleotide alone or a CpG oligonucleotide and monophosphoryl lipid A (MPL), where the polynucleotide expression vector is administered subcutaneously and the virus antigen is administered intranasally, and where the polynucleotide expression vector and virus antigen are administered in amounts sufficient for reducing symptoms and viral load of the RSV infection in the subject. In some embodiments, the RSV antigen is the prefusion F (pre-F) protein.

In some embodiments, the priming and/or boosting composition are administered prior to the subject's exposure to RSV. In some embodiments, the priming and/or boosting composition are administered after the subject's exposure to RSV.

In some embodiments, the method further includes the step of administering a second boost containing one or more aerosolized formulations including the respiratory pathogen antigen alone or in combination with the polynucleotide expression vector. The step of administering the polynucleotide expression vector and the respiratory pathogen protein in the first boost, such as RSV pre-F protein, may be carried out concurrently or together in a single formulation. In some embodiments, the second boost is administered to the subject between 2 weeks and 2 months after the first boost. In certain particular embodiments, the second boost is administered to the subject between about 2 weeks, 3 weeks, or 4 weeks after the first boost.

Another aspect of the present application relates to a method to boost existing immunity to a respiratory pathogen in a subject that has been immunized against the respiratory pathogen with a vaccine that encodes or comprises a target antigen of the respiratory pathogen.

The method comprises the step of administering to the subject an effective amount of a boosting composition comprising the target antigen by mucosal administration, e.g., intranasal administration, wherein the boosting composition enhances the preexisting immunity to the respiratory pathogen. In some embodiments, the boosting composition is administered multiple times.

Different frequencies and intervals from administering the priming composition and boosting compositions may be used. The first boosting composition can be administered between 2-8 weeks, preferably 4-6 weeks, or more preferably about 2-4 weeks following the administration of the priming composition. In one embodiment, the first boosting composition is administered about 7 to about 18 days after the priming composition. In another embodiment, the first boosting composition is administered about 10 to about 16 days after the priming composition.

Different intervals between boosting compositions (e.g. the first boosting composition and the second boosting composition) may also be used and they may be the same as or different from the interval between administration of the priming composition and the first boosting composition. For instance, the second boosting composition (or subsequent boosting composition) can be administered 2-8 weeks, preferably 4-6 weeks, or more preferably about 2-4 weeks following the administration of the first boosting composition or previous boosting composition. In one embodiment, the second boosting composition is administered about 7 to about 18 days after the first boosting composition (or previous boosting composition). In another embodiment, the second boosting composition is administered about 10 to about 16 days after the first boosting composition (or previous boosting composition).

The compositions and methods of the present application are useful for prophylactic vaccination (i.e. inducing a protective immune response in a subject). As used herein, a “protective immune response” or “protective immunity” refers to immunity or eliciting an immune response against an infectious agent, which is exhibited by a vertebrate (e.g., a human), that prevents or protects against infection and diseases. A protective immune response that prevents or protects against the appearance of disease symptoms will reduce or stop the spread of the infectious agent in a population by reducing viral shedding. In some embodiments, the protective immunity induced by the vaccine of the present invention is a sterilizing immunity. “Sterilizing immunity” is an immune response that eliminates or prevents an infection or is rapidly cleared, leaving no detectable trace. Prophylactic administration of the priming and boost compositions of the present application serve to prevent or ameliorate any subsequent infection. The priming and boost compositions of the present application of the present applications should are administered in prophylactically effective amounts to induce the production of broadly neutralizing antibodies and provide both cellular, humoral responses and protective immunity against challenge by a respiratory pathogen infection.

The compositions and methods of the present application provide a means for modulating immune responses such that a desired immune response biased towards a T helper type 1 (Th1) response may be elicited in an animal. The phrase “biased towards” refers to the situation where the observed immune response is closer to a Th1 but not tor T helper type 2 (Th2) response as compared to the response before immunization. In other embodiments, immunization may result in a mixed or balanced Th1 and Th2 response or a weaker Th1 response.

Most immune responses are regulated by T lymphocytes, which initiate and shape the nature of the response. As immune responses mature, CD4+T lymphocytes can become polarized towards T helper type 1 (Th1) or T helper type 2 (Th2) immune responses. As used herein, the phrases “T helper type 1 response” and “Th1 response” are used interchangeably to refer to a range of host animal responses including one or more, usually all the characteristics listed in the middle column of Table I above. These characteristics include a ratio of IgGI:IgG2a of no greater than 0.5; increased IFN-y (and other Th1 cytokines) secretion by T helper I cells and decreased IL-10 and IL4 (and other Th2 cytokines) secretion by T helper 2 cells; and high CTL activity. Similarly, as used herein, the phrases “T helper type 2 response” and “Th2 response” are used interchangeably to refer to a range of host animal responses including one or more, usually all the characteristics listed in the right column of Table I below. These characteristics include a ratio of IgGI:IgG2a of no less than 2.0; decreased IFN-y (and other Th1 cytokines) secretion by T helper 1 cells and increased IL-10 and IL-4 (and other Th2 cytokines) secretion by T helper 2 cells; and low or absent CTL activity.

The hallmark of Th1 and Th2-type responses is the predominant pattern of cytokines that are present. Th1 responses are characterized by high levels of IFN-y and low levels of IL-4 and IL-10, while Th2 responses are characterized by low levels of IFN-y and high levels of IL-4 and IL-10. These cytokines play an important role in determining the functional capabilities of the T cells. Th2-type responses lead to the preferred production of antibodies of the IgGI subclass, with little or no generation of CTLs. Th1-type responses lead to the preferred production of antibodies of the IgG2a subclass and induction of CTLs that can effectively kill cells infected with viruses or other organisms.

Table 1 below summarizes the immunological characteristics of Th1 and Th2 polarized immune responses. Th1 polarized responses are typically generated during infections with viruses or bacteria. In contrast, Th2 polarized responses are often observed in parasitic infections, in allergic responses, and by conventional alum-based intramuscularly delivered protein vaccines that are used in humans. Genetics can also determine the type of immune responses generated. For example, Th1 responses predominate in the C57BL/6 strain of mouse, while Th2 responses predominate in the BALB/c strain of mouse. Immune responses may also consist of both Th1 and Th2 components, affording protection by both humoral and cell mediated arms of the immune response. Direct determination of the frequencies of cytokine producing cells can be accomplished using ELISPOT (Enzyme Linked Immunosorbent SPOT assays) or by immunofluorescence staining to reveal intracellular cytokine production. Serum IgGI:IgG2a ratios are also widely accepted and followed criteria to determine T helper types (Table 1). An IgG1 to IgG2a ratio for balanced Th1 and Th2 response would be between 0.5 and 2.0.

TABLE 1
A Characteristics of Th1 and Th2 polarized T cell responses.

| | Th1-type immune | Th2-type immune |
|---|---|---|
| Immune Responses | responses | responses |
| Humoral immunity | Serum IgG1/IgG2a < 0.5 | Serum IgG1/IgG2a > 2.0 |
| T cell cytokine | ↑ IFN-γ; | ↑ IL-10 and IL-4; |
| secretion | ↓ IL-10 and IL-4 | ↓ IFN-γ |
| CTL | High | Low or absent |
| Prototypical mouse | C57BL/6 | Balb/c |
| strains | | |

### Methods of Administration

Appropriate doses for use in human and non-human mammals can be determined by those of ordinary skill in the art. The precise dosage can depend on the type of vector used, the promoter, the level of expression of antigen, administration methods, and the type and level of codon-optimization of the antigen nucleotide sequence.

The initial boost administration and the subsequent boost administrations may use the same or different amounts of the protein antigen. Moreover, the boost administrations may be administered via the same or different routes. In some embodiments, the boost compositions also contain the antigen-encoding polynucleotide vector of the priming composition.

The specific dose of the polynucleotide expression vector in the priming composition and the protein antigen in the boosting composition of the present application may be determined by their efficacy in experimental animal models, the particular circumstances of the individual patient including the size, weight, age and sex of the patient, the nature and stage of the disease, the aggressiveness of the disease, and the route of administration of the composition. A “unit dose” as used herein refers to the amount(s) of physically discrete vaccine(s) administered at a given point in time for the subject to be vaccinated, where each unit dose contains a predetermined quantity of protein antigen-encoding polynucleotides or protein antigens, individually or collectively, to produce a desired level of protective immunity or therapeutic efficacy. The unit dose administered at a given point in time may be comprised of multiple injections (e.g., 2, 3, 4, 5, 6 etc.) collectively totaling the amount of the unit dose.

The priming and boosting compositions are administered in amounts sufficient to be prophylactically or therapeutically effective. It is especially advantageous to formulate the pharmaceutical composition of the present application in dosage unit form for ease of administration and uniformity of dosage. The exact quantity may vary considerably depending on the species and weight of the animal being immunized, the route of administration, the potency and dose of the priming and boosting compositions, the nature of the disease state being treated or protected against, the capacity of the subject's immune system to produce an immune response and the degree of protection or therapeutic efficacy desired. Based upon these variables, a medical or veterinary practitioner can readily be able to determine the appropriate dosage level.

As a general proposition, the amounts of the polynucleotide expression vector in the priming composition and the protein antigen in the booting compositions may be formulated in a unit dose ranging from about 1 ng/kg body weight/day to about 100 mg/kg body weight/day whether by one or more administrations. In more particular embodiments, the polynucleotide expression vector in the priming composition and/or the protein antigen in the booting composition is administered in weight range from about 1 ng/kg body weight/day to about 1 μg/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 μg/kg body weight/day to about 10 μg/kg body weight/day, 1 μg/kg body weight/day to about 100 μg/kg body weight/day, 10 μg/kg body weight/day to about 100 μg/kg body weight/day, 10 μg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day.

In other embodiments, the polynucleotide expression vector in the priming composition and/or the protein antigen in the boosting composition is administered at a dosage range of 1 ng-10 ng per injection, 10 ng-100 ng per injection, 100 ng-1 μg per injection, 1 μg-10 μg per injection, 10 μg-100 μg per injection, 100 μg-1 mg per injection, 1 mg-10 mg per injection, 10 mg-100 mg per injection, and 100 mg-1000 mg per injection.

In some embodiments, the polynucleotide expression vector in the priming composition and/or the protein antigen in the boosting composition is administered in a range from about 1 ng/kg to about 100 mg/kg. In more particular embodiments, each of these components may be administered in a range from about 1 ng/kg to about 10 ng/kg, about 10 ng/kg to about 100 ng/kg, about 100 ng/kg to about 1 μg/kg, about 1 μg/kg to about 10 μg/kg, about 10 μg/kg to about 100 μg/kg, about 100 μg/kg to about 1 mg/kg, about 1 mg/kg to about 10 mg/kg, about 10 mg/kg to about 100 mg/kg, about 0.5 mg/kg to about 30 mg/kg, and about 1 mg/kg to about 15 mg/kg.

In certain particular embodiments, the polynucleotide expression vector in the priming composition and/or the protein antigen in the boosting composition is administered at about 0.0006, 0.001, 0.003, 0.006, 0.01, 0.03, 0.06, 0.1, 0.3, 0.6, 1, 3, 6, 10, 30, 60, 100, 300, 600 and 1000 mg/day.

In some embodiments, the total amount of the polynucleotide expression vector in the priming composition and/or the protein antigen in the boosting composition in a unit dose is in a range between 1 μg/kg to 1 mg/kg, e.g., from 5 μg/kg-500 mg/kg, 10 μg/kg-250 μg/kg, or 10 μg/kg-170 μg/kg. Further, in some embodiments, the protein antigen in the boosting composition is present in a unit dose of the composition in a range between 5 μg/kg-500 μg/kg, e.g., 10-100 μg/kg.

To improve the effectiveness of the boosting composition of the present application, multiple injections can be used for therapy or prophylaxis over extended periods of time. In some embodiments, the boosting composition is administered to the subject in multiple administrations with an interval of 1-26 weeks. In some embodiments, the first boosting composition is administered about 1 to 12 weeks following the administration of the priming composition, and the second boosting composition is administered about 1 to 12 weeks following the first boosting.

In some embodiments, the boosting composition is administered multiple times, e.g., between two to six times, e.g., three, four, or five times. Further, the boosting composition may be administered at various times after the initial administration. For example, in one embodiment, the boosting composition is administered every 3 weeks, every 4 weeks, every 6 weeks, every 8 weeks or combination thereof. In another embodiment, the boosting composition is administered at 0, 4 and 12 weeks. The treating physician can determine whether to increase or decrease vaccinations based on a patient's response, including e.g., evaluation of immune responses, viral loads etc.

Toxicity and efficacy of the composition of the present application can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index can be expressed as the ratio LD50/ED50. Compositions exhibiting large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

### Routes of Administration

Any suitable route or mode of administration can be employed for providing a subject with a therapeutically or prophylactically effective dose via a mucosal route (e.g., nasal, sublingual, buccal, rectal, vaginal). Administration of the compositions by the routes of administration described herein may involve the use of a variety electronic/mechanical devices for dispensing the priming and boosting compositions described herein. For example, for embodiments in which the priming composition is administered by injection, devices, such as e.g., syringes equipped with needles, autoinjectors or pen-injectors may be used to administer the priming composition. The devices may deliver the boosting compositions by passive means requiring the subject to inhale the formulation into the nasal cavity, upper and lower respiratory tracts. Alternatively, the devices may actively deliver the boosting compositions by pumping or spraying a dose into the nasal cavity. The boosting compositions may be delivered into one or both nostrils by one or more such devices by using e.g., one device or two devices per subject (one device per nostril).

The compositions of the present application can have multiple modes and routes of administration. In preferred embodiments, the polynucleotide vector compositions are administered intramuscularly (IM) or intradermally (ID). By either route, they can be administered by needle injection, gene gun, or needleless jet injection (e.g., Biojector™ (Bioject Inc., Portland, OR) and/or microneedle patch. In some cases, IM delivery can also be accomplished by electrotransfer (e.g., applying a series of electrical impulses to muscle immediately after immunization). Other modes of administration include oral, intravenous, intraperitoneal, intrapulmonary, intravitreal, and subcutaneous inoculation.

In preferred embodiments, the protein immunogens are administered by a mucosal route. Mucosal routes of administration include e.g., intranasal, ocular, oral, vaginal, or rectal, and topical routes. Administration by mucosal routes entry through mucosal surfaces may be carried out by variety of methods including the use of inhalants, nasal spray, nose-drops, suppositories, microspheres, and microparticles. In some embodiments, the immunogens may be encapsulated in poly(lactide-co-glycolide) (PLG) microparticles by a solvent extraction technique, such as the ones described in Jones et al., Infect. Immun., 64:489, 1996; and Jones et al., Vaccine, 15:814, 1997. For example, the immunogens can be emulsified with PLG dissolved in dichloromethane, and this water-in-oil emulsion is emulsified with aqueous polyvinyl alcohol (an emulsion stabilizer) to form a (water-in-oil)-in-water double emulsion. This double emulsion is added to a large quantity of water to dissipate the dichloromethane, which results in the microdroplets hardening to form microparticles. These microdroplets or microparticles are harvested by centrifugation, washed several times to remove the polyvinyl alcohol and residual solvent, and finally lyophilized.

### Formulation

Exemplary carriers or excipients include but are not limited to, calcium carbonate, calcium phosphate, various sugars, starches, cellulose derivatives, gelatin, polymers such as polyethylene glycols, water, saline, isotonic aqueous solutions, phosphate buffered saline, dextrose, 0.3% aqueous glycine, glycerol, ethanol and the like, as well as combinations thereof. In many cases, it will be preferable to include isotonic agents, for example, sugars, polyalcohols such as mannitol, sorbitol, or sodium chloride in the composition, or glycoproteins for enhanced stability, such as albumin, lipoprotein and globulin. Pharmaceutically acceptable carriers may further comprise minor amounts of auxiliary substances such as wetting or emulsifying agents, preservatives or buffers, which enhance the shelf life or effectiveness of the therapeutic agents. In certain embodiments, the pharmaceutically acceptable carrier comprises serum albumin.

Formulation characteristics that can be modified include, for example, pH and osmolality. For example, it may be desired to achieve a formulation that has a pH and osmolality similar to that of human blood or tissues to facilitate the formulation's effectiveness when administered parenterally.

Buffers are useful in the present disclosure for, among other purposes, manipulation of the total pH of the pharmaceutical formulation (especially desired for parenteral administration). A variety of buffers known in the art can be used in the present formulations, such as various salts of organic or inorganic acids, bases, or amino acids, and including various forms of citrate, phosphate, tartrate, succinate, adipate, maleate, lactate, acetate, bicarbonate, or carbonate ions. Particularly advantageous buffers for use in parenterally administered forms of the presently disclosed compositions in the present disclosure include sodium or potassium buffers, including sodium phosphate, potassium phosphate, sodium succinate and sodium citrate.

Sodium chloride can be used to modify the tonicity of the solution at a concentration of 0-300 mM (optimally 150 mM for a liquid dosage form). Cryoprotectants can be included for a lyophilized dosage form, principally 0-10% sucrose (optimally 0.5-1.0%).

Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%).

In one embodiment, sodium phosphate is employed in a concentration approximating 20 mM to achieve a pH of approximately 7.0. A particularly effective sodium phosphate buffering system comprises sodium phosphate monobasic monohydrate and sodium phosphate dibasic heptahydrate. When this combination of monobasic and dibasic sodium phosphate is used, advantageous concentrations of each are about 0.5 to about 1.5 mg/ml monobasic and about 2.0 to about 4.0 mg/ml dibasic, with preferred concentrations of about 0.9 mg/ml monobasic and about 3.4 mg/ml dibasic phosphate. The pH of the formulation changes according to the amount of buffer used.

Depending upon the dosage form and intended route of administration it may alternatively be advantageous to use buffers in different concentrations or to use other additives to adjust the pH of the composition to encompass other ranges. Useful pH ranges for compositions of the present disclosure include a pH of about 2.0 to a pH of about 12.0.

In some embodiments, the pharmaceutical compositions of the present application are stored as a lyophilized powder under aseptic conditions and combined with a sterile aqueous solution prior to administration. The aqueous solution can contain pharmaceutically acceptable auxiliary substances as required to approximate physical conditions, such as pH adjusting and buffering agents, tonicity adjusting agents and the like, as discussed above. Alternatively, the pharmaceutical composition of the present application can be stored as a suspension, preferable an aqueous suspension, prior to administration.

The pharmaceutical composition of the present disclosure is formulated to be compatible with its intended route of administration. Examples of routes of administration include parenteral, e.g., intrathecal, intra-arterial, intravenous, intradermal, subcutaneous, oral, transdermal (topical) and transmucosal administration. Solutions or suspensions used for parenteral, intradermal, or subcutaneous application can include the following components: a sterile diluent such as water for injection, saline solutions, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral compositions may be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. Injectable compositions should be sterile and should be fluid to the extent that easy syringability exists. The compositions should be stable under the conditions of manufacture and storage and should be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the nucleic acid/protein immunogens in the required amount in an appropriate solvent with one or a combination of ingredients enumerated above, as required, followed by filtered sterilization. Generally, dispersions are prepared by incorporating the nucleic acid/protein immunogens into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active nucleic acid and/or protein immunogen(s) plus any additional desired ingredient from a previously sterile-filtered solution thereof.

Systemic administration can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

Transmucosal administration can be accomplished through the use of nasal sprays or suppositories. For transdermal administration, the pharmaceutical compositions are formulated into ointments, salves, gels, or creams as generally known in the art.

### Kits

In another aspect, the present application provides a kit for eliciting a prophylactic or therapeutic immune response against a respiratory pathogen in a subject (e.g. mammal or human). In some embodiments, the kit comprises (a) a priming composition comprising a polynucleotide vector encoding an antigenic peptide of the respiratory pathogen, formulated for subcutaneous or intramuscular injection; (b) a boosting composition comprising the antigenic peptide of the respiratory pathogen, formulated for mucosal delivery.

In one embodiment, the present application provides a mucosal vaccine kit containing: (a) a polynucleotide vector containing a respiratory virus gene operatively linked to a promoter for expressing a respiratory pathogen protein encoded by the gene, optionally where the priming composition is formulated for subcutaneous injection; (b) one or more boost composition(s), each containing a respiratory pathogen protein and one or more adjuvants, optionally where the boost composition(s) are formulated for intranasal administration, and (c) one or more pharmaceutically acceptable carrier(s), where the polynucleotide vector in the priming composition encodes the same virus protein present in the one or more boost compositions.

In one embodiment, the present application provides an RSV mucosal vaccine kit containing: (a) a polynucleotide vector comprising an RSV pre-F gene operatively linked to a promoter for expressing the full-length, membrane bound RSV pre-F protein, optionally formulated in a priming composition for intramuscular injection; (b) the extracellular domain of the RSV pre-F protein in (a), optionally formulated in a boosting composition for intranasal delivery; (c) CpG 7909 alone or CpG 7909 and monophosphoryl lipid A (MPL), and (d) no, one or more pharmaceutically acceptable carriers.

In some embodiments, the first immunizing component is formulated for intramuscular administration and the second immunizing component is formulated for mucosal administration, for instance intranasally, sublingually, buccally or via inhalation.

In some embodiments, the kit further includes diagnostic reagents to monitor immune responses to the immunogenic compositions in the subject, including polynucleotides and antigens present in the immunogenic compositions and detection reagents therefore. In some embodiments, the kit may include one or more adjuvants described in the disclosure herein.

In some embodiments, the kit may further include one or more carriers and excipients described in the disclosure herein and formulated for use with the polynucleotide or protein compositions for immunization or they may be separately packaged.

In some embodiments, the kit further comprises one or more delivery devices for administering the priming compositions and/or boosting compositions as described above.

Additionally, the kit may include instructions for use, including instructions for prophylactic administration (e.g., DNA or/protein with/out adjuvant vaccinations and boosts), including suggested dosages and/or modes of administration, as described herein.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: Study Design for Testing the Immunogenicity, Protective Efficacy and Safety of Mucosal RSV Vaccines in Cotton Rat Models

Priming animals with a customized and optimized DNA vaccine vector is predicted to determine the ultimate safety outcome of mucosal RSV vaccine formulations, although various administration formulations, such as pre-F+CpG vaccine alone may score some pulmonary pathology post live RSV challenge when applied alone. MPL was believed to be able to alleviate the adverse effect.

Inclusion of MPL in the pre-F+CpG+MPL groups may not be necessary to avoid induction of lung histopathology, when an optimized DNA vector is used. It was hypothesized that one DNA prime and one round of CpG adjuvanted boost may be sufficient for best neutralization antibody responses and nasal protection.

### Overall Study Design

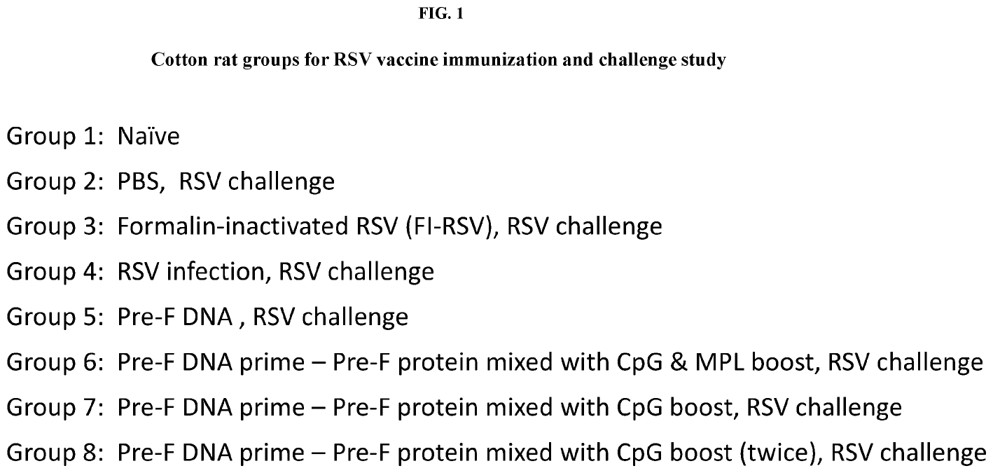

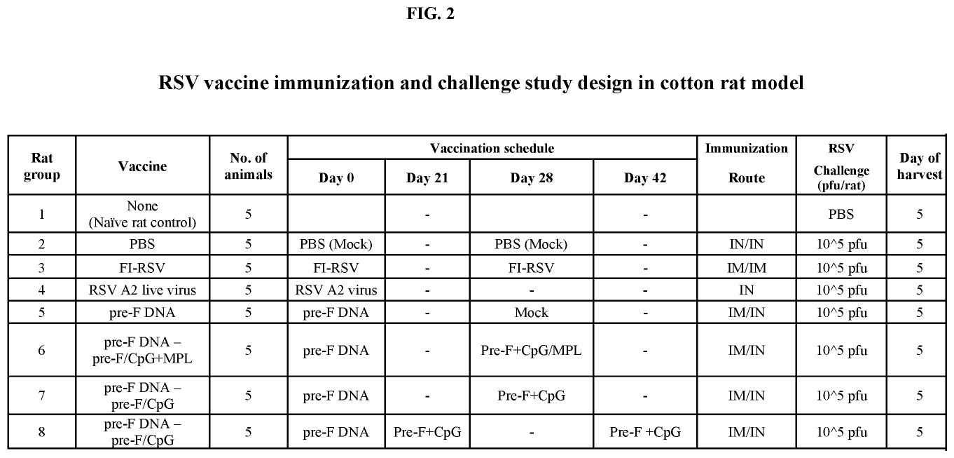

A summary of the animal groups in this study is shown in FIG. 1. FIG. 2 summarizes the design of the RSV vaccine immunization and challenge study in the cotton rat model.

Efficacy and safety of RSV pre-fusion F protein vaccine candidates were evaluated in which an intramuscular pre-FL1fn DNA prime (SEQ ID NO: 1) (which expresses a full-length RSV A2 strain post-fusion F gene using humanized codons for expression which is 565 aa long (SEQ ID NO: 2) without 9 aa fusion peptide (SEQ ID NO: 3) in a NTC8385-VA1 DNA vector (SEQ ID NO: 4), where the full-length RSV A2 strain pre-fusion F gene (DS-Cavl) using humanized codons for expression is 574 aa long (SEQ ID NO: 5) without the 9 aa fusion peptide deletion) was followed by an intranasal boost with pre-sFfs protein (SEQ ID NO:6) (a 513 aa long, truncated RSV pre-fusion F protein with the 9 aa fusion peptide not deleted) formulations adjuvanted with CpG and MPL (Group 6) or CpG alone (Group 7, 8) in the cotton rat (Sigmodon hispidus) RSV A/A2 challenge model. Also provided: codon optimized nucleotide sequence of RSV-F-A2 (SEQ ID NO: 7).

Pre-F/CpG boosts were administered twice at 3 and 6 weeks after priming with pre-F DNA (Group 8) or once 4 weeks after priming (Group 7). A pre-F/CpG/MPL boost was administered 4 weeks after priming. See FIG. 2.

As shown in FIG. 2, a control group of animals was immunized intramuscularly with pre-F DNA and not boosted (Group 5). A primary infection control group was mock immunized with PBS and then infected with RSV A/A2 (Group 2). A normal rat control was mock challenged with PBS (Group 1). A secondary infection control group was infected with RSV A/A2 and re-infected seven weeks later (Group 4). A vaccine-enhanced disease control group was immunized with FI-RSV twice with an interval of four weeks and infected with RSV three weeks after the second immunization (Group 3).

### Vaccine Formulations and Adjuvant Testing

The CpG adjuvant 7909 used in this study was obtained from NIH supported BEI resources.

RSV pre-fusion F expressing DNA: Pre-F DNA vaccine was formulated and administered at 50 μg/100 μl/animal (25 μg/50 μl/leg, IM in left and right hind legs) to prime animals. Booster formulations: Pre-F+CpG (15 μg pre-F+20 μg CpG, 25 μI/animal; intranasal (IN), pre-F+CpG+MPL (15 μg pre-F+20 μg CpG+5 μg MPL/25 μI/animal, IN).

### Animals:

Fifty (50) inbred, 6-8 weeks old, Sigmodon hispidus female and male cotton rats (source: Sigmovir Biosystems, Inc., Rockville MD) were maintained and handled under veterinary supervision in accordance with the National Institutes of Health guidelines and Sigmovir Institutional Animal Care and Use Committee's approved animal study protocol (IACUC Protocol #15). Each group of animals included 3 females (the first three animals in each group) and 2 males (the last 2 animals in each group). Cotton rats were housed in clear polycarbonate cages and provided with standard rodent chow (Harlan #7004) and tap water ad lib. Eartags were used to identify the animals in the study.
Bleeding: Retro-orbital sinus bleed
Route of infection/priming: Intranasal (i.n.) inoculation

### Viruses:

Respiratory Syncytial Virus strain A/A2 (RSV A/A2) (ATCC, Manassas, VA) was propagated in HEp-2 cells after serial plaque-purification to reduce defective-interfering particles. A pool of virus designated as hRSV A/A2 Lot #092215 SSM containing approximately 3.0×10⁸ pfu/mL in sucrose stabilizing media was used in this in vivo experiment. This stock of virus is stored at −80° C. and has been characterized in vivo in the cotton rat model and validated for upper and lower respiratory tract replication.

Endpoint assay design:

| Endpoint Assays | Samples | Storage |
|---|---|---|
| RSV viral titrations | Lung homogenates | −80° C. |
| | Nose homogenates | |
| Pulmonary histopathology | H&E lung slides | RT |
| RSV neutralizing Ab | Serum | −20° C. |
| (RSV A/A2) (d0, 28 and 49) | | |
| RSV A/A2 serum IgG ELISA | Serum | −20° C. |
| (d0, 21 (G&J), 28, 42 (G&J), 49) | | |
| (4-dilution format) | | |
| qPCR | Lung RNA | −80° C. |
| RSV A/A2 NS-I gene; | | |
| IL-4, 11-2, IFN-y | | |

### Endpoint Assay Methods

### 1. Lung and Nose Viral Titrations

Lung and nose homogenates are clarified by centrifugation and diluted in EMEM. Confluent HEp-2 monolayers are infected in duplicates with diluted homogenates in 24 well plates. After one hour incubation at 37° C. in a 5% CO2 incubator, the wells are overlayed with 0.75% Methylcellulose medium. After 4 days of incubation, the overlay is removed, and the cells are fixed with 0.1% crystal violet stain for one hour and then rinsed and air dried. Plaques are counted and virus titer is expressed as plaque forming units per gram of tissue. Viral titers are calculated as geometric mean±standard error for all animals in a group at a given time.

### 2. RSV Neutralizing Antibody Assay (60% Reduction)

Heat inactivated sera samples are diluted: IO with EMEM and serially diluted further I:4. Diluted sera samples are incubated with RSV (25-50 PFU) for I hour at room temperature and inoculated in duplicates onto confluent HEp-2 monolayers in 24 well plates. After one hour incubation at 37° C. in a 5% CO2 incubator, the wells are overlayed with 0.75% Methylcellulose medium. After 4 days of incubation (6 days for RSV B), the overlay is removed and the cells are fixed with 0.1% crystal violet stain for one hour and then rinsed and air dried.

The corresponding reciprocal neutralizing antibody titers are determined at the 60% reduction end-point of the virus control using the statistics program “plqrd.manual.entry”. The geometric means±standard error for all animals in a group at a given time are calculated.

### 3. Pulmonary Histopathology

Lung are dissected and inflated with 10% neutral buffered formalin to their normal volume, and then immersed in the same fixative solution. Following fixation, the lungs are embedded in paraffin, sectioned and stained with hematoxylin and eosin (H&E). Four parameters of pulmonary inflammation are evaluated: peribronchiolitis (inflammatory cell infiltration around the bronchioles), perivasculitis (inflammatory cell infiltration around the small blood vessels), interstitial pneumonia (inflammatory cell infiltration and thickening of alveolar walls), and alveolitis (cells within the alveolar spaces). Slides are scored blind on a 0-4 severity scale. The scores are subsequently converted to a 0-100% histopathology scale.

### 4. Real-Time PCR

Total RNA is extracted from homogenized tissue or cells using the RNeasy purification kit (QIAGEN). One μg of total RNA is used to prepare cDNA using Super Script II RT (Invitrogen) and oligo dT primer (1 μl, Invitrogen). For the real-time PCR reactions the Bio-Rad iQTM SYBR Green Supermix is used in a final volume of 25 μl, with final primer concentrations of 0.5 μM. Reactions are set up in duplicates in 96-well trays. Amplifications are performed on a Bio-Rad iCycler for 1 cycle of 95° C. for 3 min, followed by 40 cycles of 95° C. for 10 seconds (s), 60° C. for 10 s, and 72° C. for 15 s. The baseline cycles and cycle threshold (Ct) are calculated by the iQ5 software in the PCR Base Line Subtracted Curve Fit mode. Relative quantitation of DNA is applied to all samples. The standard curves are developed using serially-diluted cDNA sample most enriched in the transcript of interest (e.g., lungs from 6 hours post RSV infection of FJ-RSV-immunized animals). The Ct values are plotted against log 10 cDNA dilution factor. These curves are used to convert the Ct values obtained for different samples to relative expression units. These relative expression units are then normalized to the level of—actin mRNA (“housekeeping gene”) expressed in the corresponding sample. For animal studies, mRNA levels are expressed as the geometric mean±SEM for all animals in a group at a given time.

### Example 2: Immunogenicity, Protective Efficacy and Safety of Mucosal RSV Vaccines in Cotton Rat Models

### 1. Vaccine Immunogenicity Before Challenge

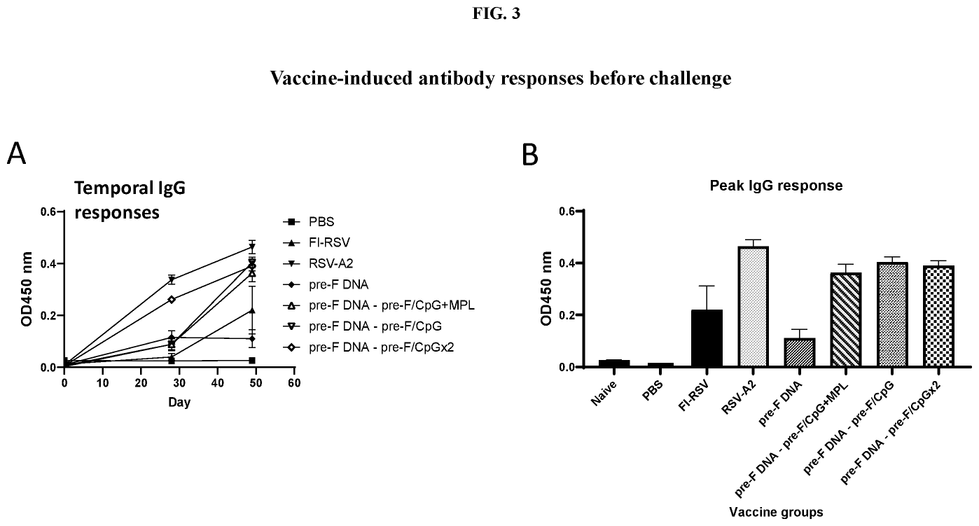

As shown in FIG. 3, Pre-F DNA prime only elicited a low level of anti-RSV F protein specific serum IgG. Two rounds of FI-RSV administration raised moderated level of anti-RSV F protein specific serum IgG. In contract, three groups of animals primed with pre-F DNA and boosted with three different pre-F formulations/frequencies generated high levels of anti-RSV F protein specific serum IgG as effective as that of the live RSV A2 infection induced. The three prime and boost strategies represented a strong, synergistic immune responses, which is correlated well with the greatly increased serum neutralizing antibody titers and superb nasal protection results.

### Lung Viral Titers

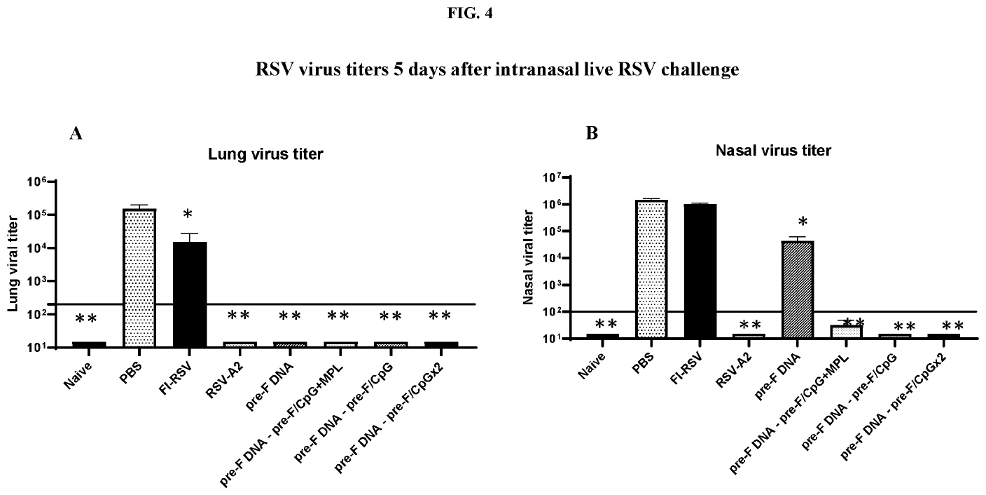

RSV A/A2 load in the lungs of cotton rats was evaluated 5 days after intranasal RSV challenge. As shown in FIG. 4, panel A, RSV-infected animals mock-immunized with PBS (Group 2) showed a titer of 5.1 Log10 PFU/g tissue in the lungs and were used for comparison to all other RSV-infected groups. Formalin-inactivated RSV (FI-RSV) immunization (Group 3) moderately, but significantly reduced lung RSV titer to 3.7 Log10 PFU/g tissue. No virus was detected in the lungs of animals infected with RSV twice (Group 4). Further, no virus was detected in the lungs of any group of animals primed with pre-F DNA without boosting (Group 5) or in groups of animals primed with pre-F DNA and boosted with various vaccine combinations (Groups 6, 7, 8; p<0.05, compared to Group 2).

### Nose Viral Titers

RSV A/A2 load in the nose of cotton rats was evaluated 5 days after intranasal RSV challenge. The result of this analysis is shown in FIG. 4, panel B. RSV-infected animals mock-immunized with PBS (Group 2) showed a titer of 6.2 Log10 PFU/g virus in the nose and were used for comparison to all other RSV-infected groups. FI-RSV immunization (Group 3) had no effect on RSV load in the nose (5.99 Log10 PFU/g). No virus was detected in the nose of animals infected with RSV twice (Group 4). Immunization with pre-F DNA alone reduced RSV titer in the nose by 2 Log10 PFU/g (Group 5). Immunization with pre-F DNA followed by a boost with pre-F adjuvanted with CpG/MPL (Group 6) or CpG administered once (Group 7) resulted in near complete (single plaques detectable in 1/5 animals in each group) protection in the nose. Boosting of Pre-F DNA-immunized animals with pre-F/CpG twice resulted in sterilizing immunity in the nose (Group 8).

### Serum RSV A/A2 Neutralizing Antibodies

Serum neutralizing antibodies (NA) against RSV A/A2 were measured in all animals prior to the start of experiment (day 0), 4 weeks after the first immunization (day 28), and three weeks later (day 49).

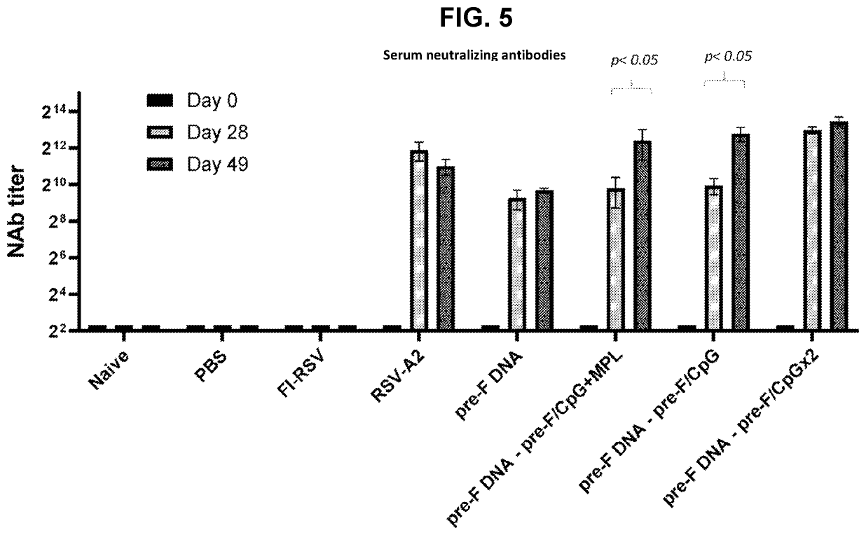

As shown in FIG. 5, animals immunized with pre-F DNA and boosted with pre-F/CpG twice (Group 8) on days 21 and 42 produced the highest NA titers on day 28 and day 49. Animals immunized with pre-F DNA and mock boosted retained comparable levels of NA on days 28 and 49. Boosting of pre-F DNA-primed animals once with pre-F CpG/MPL (Group 6) or once with CpG (Group 7) further increased neutralizing antibody titers on day 49 relative to day 28.

Two rounds of boosts with CpG-adjuvanted pre-F did not significantly enhance the NAb response beyond that of a single CpG-adjuvanted pre-F boost. All three types of boosts increased neutralizing antibodies to a level higher than in RSV-infected animals on day 49.

### Serum RSV A/A2 Binding IgG Antibodies

Serum binding IgG antibodies against RSV A/A2 F protein were measured in all animals prior to the start of experiment (day 0), 4 weeks after the first immunization (day 28), and three weeks later (day 49). As shown in FIG. 3, an increase in binding IgG was visible in animals vaccinated with FI-RSV. Animals immunized with pre-F DNA alone showed low levels of binding IgG. Animals immunized with pre-F DNA and boosted by various methods had comparable levels of binding IgG on day 49. Intranasal administration of pre-F/CpG showed boosting effect, with increases between day 21 and day 28, and day 42 and day 49. The first increase was more pronounced than the second increase.

### Lung Histopathology

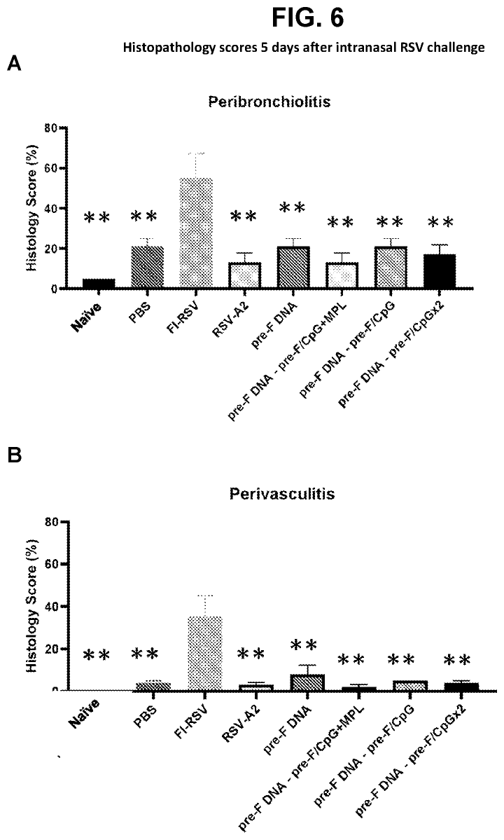

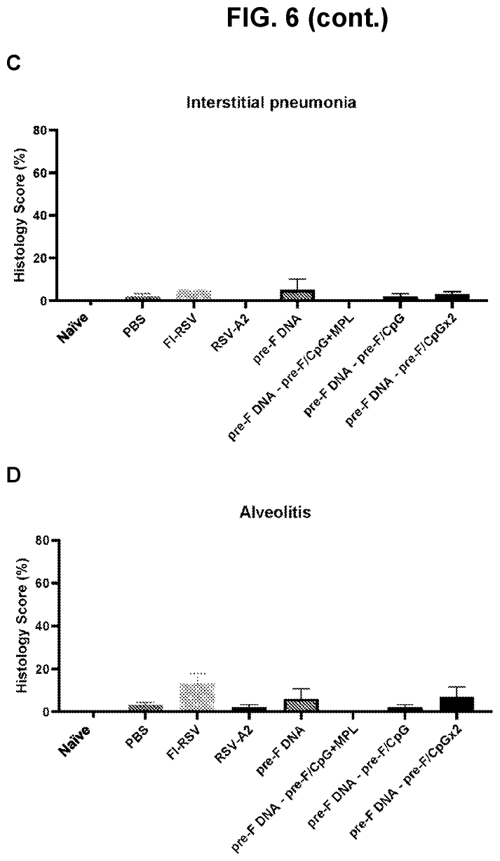

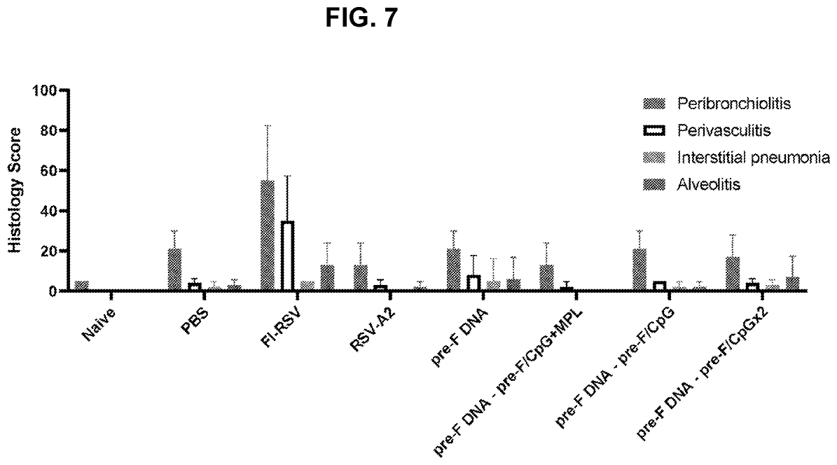

Pulmonary histopathology was evaluated in all animals 5 days after RSV A/A2 intranasal challenge. The results of this analysis are shown in FIGS. 6 and 7. RSV-infected animals mock-immunized with PBS (Group 2) or challenged with RSV twice (Group 4) had moderate levels of pathology. The highest level of pulmonary histopathology was detected in animals immunized with FI-RSV (Group 3), which exhibited significant levels interstitial inflammation and alveolitis. None of the vaccines induced pulmonary histopathology to the extent seen in FI-RSV-immunized animals. Compared to animals with primary or secondary RSV infections (Groups 2 and 4, respectively), each group of animals was immunized with pre-F DNA alone (Group 5) or further boosted with pre-DNA/CpG once (Group 7) or twice (Group 8) had slightly elevated alveolitis (FIG. 6, panel D and FIG. 7). Animals immunized with pre-F DNA and boosted once with pre-F/CpG/MPL had the lowest level of pulmonary pathology of all the vaccinated groups evaluated.

Nasal and Lung IgA and IgG Responses after Intranasal RSV Challenge

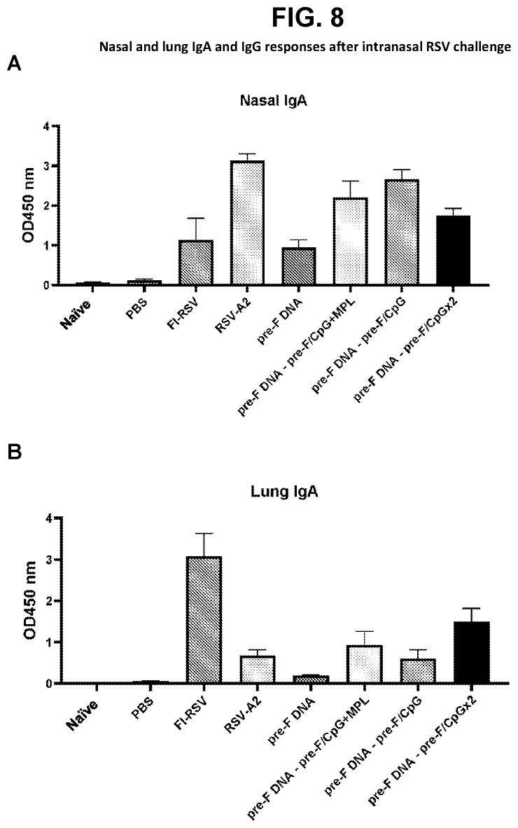

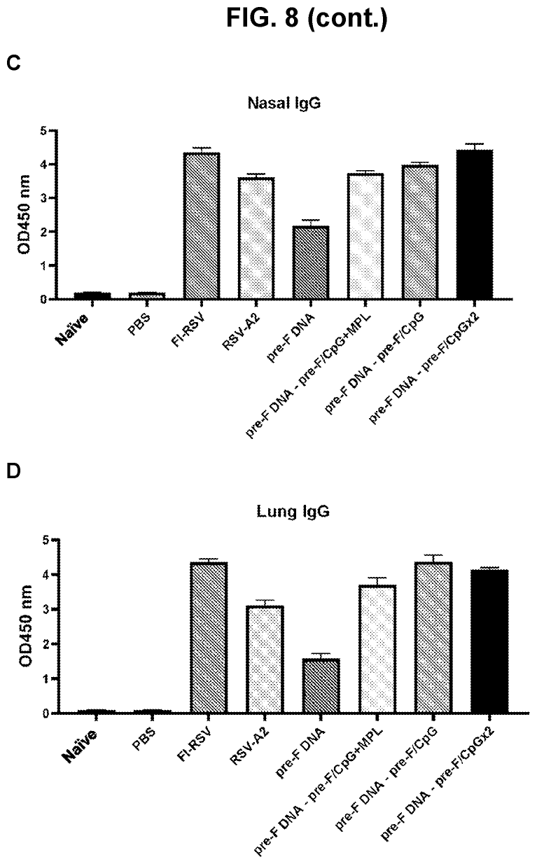

Using the supernatant of collected nose and lung tissue homogenates, nasal and lung binding IgA and IgG antibodies against RSV A/A2 F protein were measured, 5 days after live RSV virus challenge (day 54). As shown in FIG. 8, Panel A: no nasal IgAs were detected in navie and PBS groups, indicating insufficient time for primary infection to raise detectable level of IgA just after 5 days of primary virus infection. Recalled IgA responses were shown to be different among all pre-infected and vaccinated groups. By evaluation of the recalled nasal IgA 5 days post intranasal virus challenge, the pre-challenge level of mucosal IgA responses in all study groups can be indirectly compared. Only low to moderate IgA responses were detected in the FI-RSV and pre-F DNA groups. In contract, the highest IgA responses were seen in the RSV A2 infected animal group, and those three primed and boosted vaccine groups. MPL did not seem to enhance nasal IgA. Two arounds of pre-F/CpG boosting did not as further increase the overall IgA level. One single around of intranasal pre-F-CpG boost appeared to be sufficient to raise optimal level of nasal IgA as efficient as the live RSV virus infection.

As shown in FIG. 8, panel B: no lung IgAs were detected. Highest IgA responses was detected in the FI-RSV group, which could be correlated with active virus infection in this group due to low protective immunity associated with FI-RSV vaccine. Only low IgA responses were detected in the pre-F DNA alone group. Moderate, comparable levels of lung IgA were detected in the RSV A2 infected group and those three primed and boosted vaccine groups. MPL did not see to enhance lung IgA. Two arounds of pre-F/CpG boosting did not significantly increase the overall lung IgA level.

As shown in FIG. 8, panel C: no or minimal nasal IgGs were detected in naive and PBS groups, indicating insufficient time for primary infection to raise detectable level of IgG just after 5 days of primary virus infection. Recalled nasal IgG response patterns were shown to be different from the nasal IgA responses of all pre-infected and vaccinated groups. By evaluation of the recalled nasal IgG 5 days post intranasal virus challenge, the pre-challenge level of IgG responses in all study groups can be indirectly compared. Moderate IgG responses were detected in the pre-F DNA group. High nasal IgG responses were seen in the RSV A2 infected animal group, the FI-RSV vaccinated and those three primed and boosted vaccine groups. MPL did not seem to enhance nasal IgG. Two arounds of pre-F/CpG boosting did not as further increase the overall nasal IgG level. One single around of intranasal pre-F-CpG boost appeared to be sufficient to raise the highest level of nasal IgG as efficient as the live RSV virus infection.

As shown in FIG. 8, panel D: no or minimal lung IgG was detected in naive and PBS groups, indicating insufficient time for primary infection to raise detectable level of lung IgG just after 5 days of primary virus infection. Recalled lung IgG response patterns were shown to be different from the lung IgA responses of all pre-infected and vaccinated groups. The lung IgG responses appeared to be almost the same pattern of nasal IgG. By evaluation of the recalled lung IgG 5 days post intranasal virus challenge, the pre-challenge level of lung IgG responses in all study groups can be indirectly compared. Moderate IgG responses were detected in the pre-F DNA group. High lung IgG responses were seen in the RSV A2 infected group, the FI-RSV vaccinated and those three primed and boosted vaccine groups. MPL did not seem to enhance lung IgG. Two arounds of pre-F/CpG boosting did not further increase the overall lung IgG level. One single around of intranasal pre-F-CpG boost appeared to be sufficient to raise the highest level of lung IgG as efficient as the live RSV virus infection.

mRNA Expression of RSV and Cytokines in Lung Following RSV Challenge

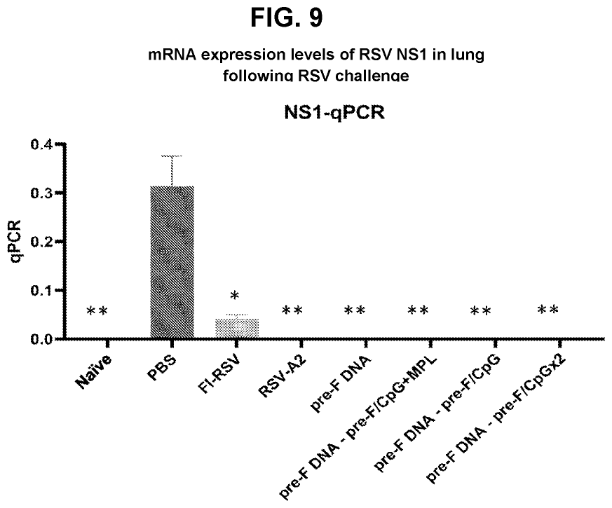

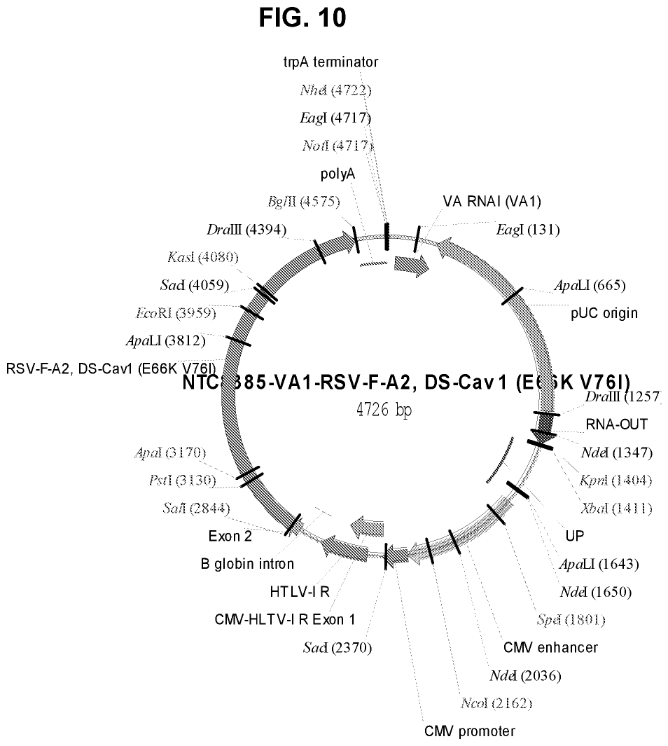

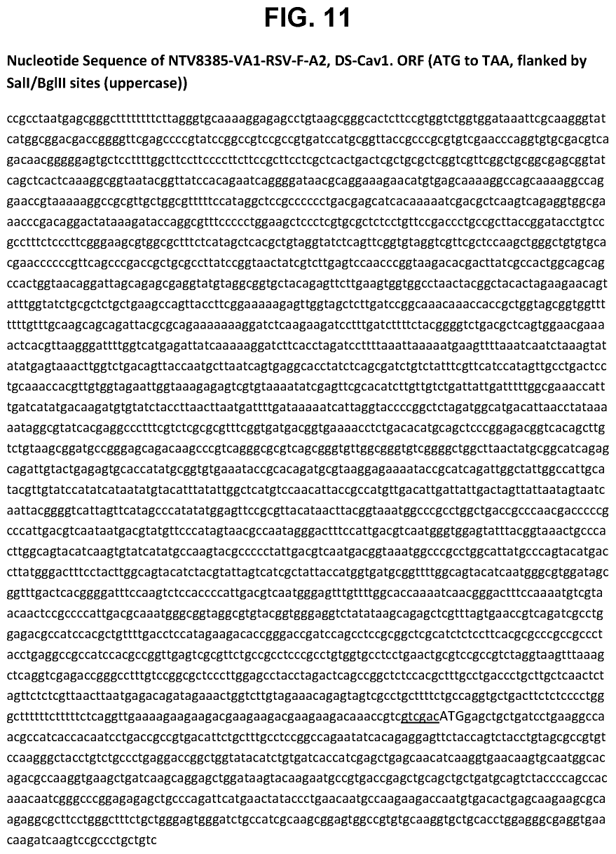

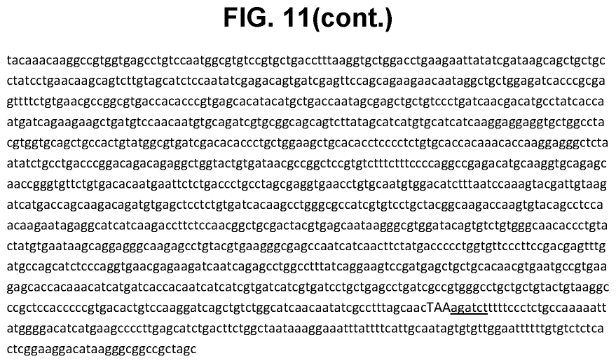

Expression of RSV NS1, IL-4, IL-2, and IFN-y mRNA was evaluated in lung samples collected on day 5 after RSV A/A2 challenge and normalized by the level of -actin mRNA in each sample. The results of this analysis are shown in FIG. 9. Expression of NS-1 mRNA was significantly reduced (undetectable) by all vaccine combinations tested. FI-RSV immunization (Groups 3) resulted in moderate reduction in lung RSV NS1mRNA level, but significantly increased IL-4 mRNA level compared to animals with primary (Group 2) and secondary (Group 4) RSV infections (data not shown). Expression of IL-2 and IFN-y mRNA expression was moderately elevated in animals with primary RSV infection (Group 2) and in RSV-infected FI-RSV-immunized animals (Group 3) (data not shown). Further, IL-4, IL-2, and IFN-y mRNA levels in all animals immunized with test vaccines did not exceed the levels seen in mock-infected animals (Group 1) or animals with primary RSV infection (Group 2) (data not shown).

### CONCLUSIONS

Efficacy and safety of an RSV pre-fusion F protein vaccine candidates based on the intramuscular pre-FL1fn DNA prime followed by the intranasal boost with pre-sFfs formulations adjuvanted with CpG and MPL or CpG alone were evaluated in the cotton rat Sigmodon hispidus model of RSV A/A2 challenge. Pre-F/CpG boosts were administered twice at 3 and 6 weeks after priming with pre-F DNA or once 4 weeks after priming. A pre-F/CpG/MPL boost was administered one 4 weeks after priming. A control group of animals was immunized intramuscularly with pre-F DNA and not boosted. The primary infection control group was mock immunized with PBS and then infected with RSV A/A2. The secondary infection control group was infected with RSV A/A2 and re-infected seven weeks later. The vaccine-enhanced disease control group was immunized with FI-RSV twice with an interval of four weeks and infected with RSV three weeks after the second immunization.

No virus was detected in the lungs of any group of animals primed with pre-F DNA with or without boosting. Immunization with pre-F DNA reduced RSV titers in the nose by 2 Log10 PFU/g. Boosting of pre-F DNA-immunized animals with pre-F/CpG administered twice resulted in sterilizing immunity in the nose. The other boosting strategies significantly improved nasal protection, with only single plaques detectable.

All vaccination strategies tested induced high levels of neutralizing antibodies (NA) and binding IgG against RSV. An increase in binding IgG was visible in animals vaccinated with FI-RSV. Animals immunized with pre-F DNA alone showed low levels of binding IgG. Animals immunized with pre-F DNA and boosted by various methods had comparable levels of binding IgG on day 49. Intranasal administration of pre-F/CpG showed boosting effect, with increases between day 21 and day 28, and day 42 and day 49. The first increase was more pronounced than the second increase.

For boosts with CpG- and/or MPL-containing pre-F, two-time boosting with CpG-adjuvanted pre-F resulted in a better NA response than one-time boosting with CpG- or CpG/MPL-adjuvanted pre-F. None of the test vaccines induced expression of IL-4, IL-2, or IFN-y mRNA beyond what was seen in primary RSV infections. Safety or histopathology profile is very important especially for respiratory infection and/or vaccine induced immunity. Our data set showed that the role of optimized pre-F DNA vector that induced a weak Th1 biased or a balanced Th1/Th2 responses in laying the safety basis and determination of the ultimate histopathology outcome of those three prime and boost vaccine regimens. Regardless the booster formulation and frequency of intranasal administration, those three prime and boost strategies were not able to change the histopathology/safety profile of what the priming vaccine established in the first place. In other words, the CpG adjuvanted protein booster vaccine was not able to induce significantly pronounced histopathology as what was reported before when it was administered alone.

Overall, pre-F DNA immunization itself induced sterilizing immunity in the lung, masking the efficacy of the various boosting formulations when evaluating pulmonary protection only. Boosting after pre-F-DNA immunization, however, was necessary and beneficial for improving nasal protection and enhancing antibody response. Pre-F/CpG administered twice showed the best results at improving nasal protection and provided sterilizing immunity in the nose when administered after pre-F DNA priming.

The booster formulation to enhance mucosal protection and IgA can be 1) protein+CpG alone or 2) protein+CPG and MPL combined. CpG alone as adjuvant seems to be sufficient to provide the best protection without compromising the safety profile. Mucosal IgA is the hallmark of mucosal immunity. By using the customized prime and boost strategy herein, this application circumvents the problem of using CpG as adjuvant without MPL. This is one of the key surprising findings of this application.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1-22. (canceled)

23. A method for treating or preventing symptoms of a respiratory and/or mucosally transmitted pathogen infection in a subject, comprising the steps of
(1) administering to the subject an effective amount of a priming composition comprising a nucleic acid based-expression system encoding a protein antigen of the respiratory and/or mucosally transmitted pathogen; and
(2) administering to the subject an effective amount of a boosting composition comprising the protein antigen of the respiratory and/or mucosally transmitted pathogen,
wherein the priming composition is administered non-mucosally, and the boosting composition is administered mucosally,
wherein the respiratory and/or mucosally transmitted pathogen is respiratory syncytial virus (RSV),
wherein the protein antigen of the respiratory and/or mucosally transmitted pathogen is RSV prefusion-F (pre-F) protein,
wherein the boosting composition comprises an adjuvant.

24. The method of claim 23, wherein the nucleic acid based-expression system comprises a DNA vector or an mRNA molecule.

25. The method of claim 23, wherein the coding sequence of the protein antigen of the respiratory and/or mucosally transmitted pathogen in the priming composition comprises a nucleotide sequence of SEQ ID NO:1, or the protein antigen of the respiratory and/or mucosally transmitted pathogen in the priming composition comprises an amino acid sequence of SEQ ID NO:5.

26. The method of claim 23, wherein the nucleic acid based-expression system comprises a nucleotide sequence of SEQ ID NO:4.

27. The method of claim 23, wherein the protein antigen of the respiratory and/or mucosally transmitted pathogen in the boosting composition comprises an amino acid sequence of SEQ ID NO:6.

28. The method of claim 23, wherein the adjuvant comprises a CpG oligonucleotide (CpG ODN) or a CpG ODN and monophosphoryl lipid A (MPL), preferably the CpG ODN is CpG 7909.

29. The method of claim 23, wherein the priming composition is administered by a non-mucosal route selected from one of the following routes consisting of intramuscularly, intravenously, intraperitoneally, intradermally, and subcutaneously, preferably intramuscularly.

30. The method of claim 23, wherein the boosting composition is administered by a mucosal route selected from one of the following routes consisting of intranasally and inhalation, preferably intranasally.

31. The method of claim 23, wherein the boosting composition is an aerosolized, or sprayed formulation.

32. The method of claim 23, wherein the boosting composition is administered about 1 to 12 weeks, such as about 4 weeks, following the administration of the priming composition.

33. The method of claim 23, wherein the boosting composition is administered multiple times, such as twice, with an interval of about 2 weeks to 2 months, such as about 3 weeks.

34. A method for treating or preventing symptoms of a RSV infection in a subject, comprising the steps of
(1) administering to the subject an effective amount of a priming composition comprising a polynucleotide expression vector encoding a RSV antigen under the control of a promoter; and
(2) administering to the subject an effective amount of a boosting composition comprising one or more aerosolized/sprayed formulations comprising the RSV antigen alone or in combination with the polynucleotide expression vector, and one or more adjuvants,
wherein the one or more adjuvants comprise a CpG ODN or a CpG ODN and monophosphoryl lipid A (MPL), and
wherein the priming composition is administered intramuscularly or subcutaneously or intrademally, and the boosting composition is administered intranasally,
wherein the RSV antigen is RSV pre-F protein.

35. A vaccine kit, comprising:
(a) a priming composition comprising a polynucleotide vector comprising a polynucleotide vector encoding a protein antigen of a respiratory or mucosally transmitted pathogen; and
(b) a boosting composition comprising the protein antigen of the respiratory or mucosally transmitted pathogen,
wherein the priming composition is formulated for intramuscular or subcutaneous or intrademal administration, and wherein the boosting composition is formulated for intranasal administration,
wherein the respiratory or mucosally transmitted pathogen is RSV,
wherein the protein antigen of a respiratory or mucosally transmitted pathogen is RSV pre-F protein.

36. The vaccine kit of claim 35, wherein the boosting composition is an aerosolized, sprayed formulation or nasal drop.


SEQUENCE LISTING (7 sequences)

SEQ ID NO: 1 - DNA, 1725 bp, synthetic construct
  misc_feature 1..1725: note = Synthetic: intramuscular pre-FL1fn DNA
  source 1..1725: mol_type = other DNA
atggagctgctgatcctgaaggccaacgccatcaccacaatcctgaccgccgtgacattctgctttgcctccggccagaatatcacagaggagttctaccagtctacctgtagcgccgtgtccaagggctacctgtctgccctgaggaccggctggtatacatctgtgatcaccatcgagctgagcaacatcaaggtgaacaagtgcaatggcacagacgccaaggtgaagctgatcaagcaggagctggataagtacaagaatgccgtgaccgagctgcagctgctgatgcagtctaccccagccacaaacaatcgggcccggagagagctgcccagattcatgaactataccctgaacaatgccaagaagaccaatgtgacactgagcaagaagcgcaagaggcgcttcctgggctttctgctgggagtgggatctgccatcgcaagcggagtggccgtgtgcaaggtgctgcacctggagggcgaggtgaacaagatcaagtccgccctgctgtctacaaacaaggccgtggtgagcctgtccaatggcgtgtccgtgctgacctttaaggtgctggacctgaagaattatatcgataagcagctgctgcctatcctgaacaagcagtcttgtagcatctccaatatcgagacagtgatcgagttccagcagaagaacaataggctgctggagatcacccgcgagttttctgtgaacgccggcgtgaccacacccgtgagcacatacatgctgaccaatagcgagctgctgtccctgatcaacgacatgcctatcaccaatgatcagaagaagctgatgtccaacaatgtgcagatcgtgcggcagcagtcttatagcatcatgtgcatcatcaaggaggaggtgctggcctacgtggtgcagctgccactgtatggcgtgatcgacacaccctgctggaagctgcacacctcccctctgtgcaccacaaacaccaaggagggctctaatatctgcctgacccggacagacagaggctggtactgtgataacgccggctccgtgtctttctttccccaggccgagacatgcaaggtgcagagcaaccgggtgttctgtgacacaatgaattctctgaccctgcctagcgaggtgaacctgtgcaatgtggacatctttaatccaaagtacgattgtaagatcatgaccagcaagacagatgtgagctcctctgtgatcacaagcctgggcgccatcgtgtcctgctacggcaagaccaagtgtacagcctccaacaagaatagaggcatcatcaagaccttctccaacggctgcgactacgtgagcaataagggcgtggatacagtgtctgtgggcaacaccctgtactatgtgaataagcaggagggcaagagcctgtacgtgaagggcgagccaatcatcaacttctatgaccccctggtgttcccttccgacgagtttgatgccagcatctcccaggtgaacgagaagatcaatcagagcctggcctttatcaggaagtccgatgagctgctgcacaacgtgaatgccgtgaagagcaccacaaacatcatgatcaccacaatcatcatcgtgatcatcgtgatcctgctgagcctgatcgccgtgggcctgctgctgtactgtaaggcccgctccacccccgtgacactgtccaaggatcagctgtctggcatcaacaatatcgcctttagcaactaa

SEQ ID NO: 2 - protein, 565 aa, synthetic construct
  REGION 1..565: note = Synthetic: The full-length RSV A2 strain post-fusion F gene using humanized codons for expression is 565 aa long with 9 aa fusion peptide
MELLILKANAITTILTAVTFCFASGQNITEEFYQSTCSAVSKGYLSALRTGWYTSVITIELSNIEKNKCNGTDAKIKLIKQELDKYKNAVTELQLLMQSTPATNNRARRELPRFMNYTLNNAKKTNVTLSKKRKRRSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHHVNAGKSTTNIMITTIIIVIIVILLSLIAVGLLLYCKARSTPVTLSKDQLSGINNIAFSN

SEQ ID NO: 3 - protein, 9 aa, synthetic construct
  REGION 1..9: note = Synthetic: 9 amino acid deletion
FLGFLLGVG

SEQ ID NO: 4 - DNA, 4726 bp, synthetic construct
  misc_feature 1..4726: note = Synthetic: nucleotide sequence of NTV8385-VAl-RSV-F-A2, DSCavl
  source 1..4726: mol_type = other DNA
ccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagatggcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacatggagctgctgatcctgaaggccaacgccatcaccacaatcctgaccgccgtgacattctgctttgcctccggccagaatatcacagaggagttctaccagtctacctgtagcgccgtgtccaagggctacctgtctgccctgaggaccggctggtatacatctgtgatcaccatcgagctgagcaacatcaaggtgaacaagtgcaatggcacagacgccaaggtgaagctgatcaagcaggagctggataagtacaagaatgccgtgaccgagctgcagctgctgatgcagtctaccccagccacaaacaatcgggcccggagagagctgcccagattcatgaactataccctgaacaatgccaagaagaccaatgtgacactgagcaagaagcgcaagaggcgcttcctgggctttctgctgggagtgggatctgccatcgcaagcggagtggccgtgtgcaaggtgctgcacctggagggcgaggtgaacaagatcaagtccgccctgctgtctacaaacaaggccgtggtgagcctgtccaatggcgtgtccgtgctgacctttaaggtgctggacctgaagaattatatcgataagcagctgctgcctatcctgaacaagcagtcttgtagcatctccaatatcgagacagtgatcgagttccagcagaagaacaataggctgctggagatcacccgcgagttttctgtgaacgccggcgtgaccacacccgtgagcacatacatgctgaccaatagcgagctgctgtccctgatcaacgacatgcctatcaccaatgatcagaagaagctgatgtccaacaatgtgcagatcgtgcggcagcagtcttatagcatcatgtgcatcatcaaggaggaggtgctggcctacgtggtgcagctgccactgtatggcgtgatcgacacaccctgctggaagctgcacacctcccctctgtgcaccacaaacaccaaggagggctctaatatctgcctgacccggacagacagaggctggtactgtgataacgccggctccgtgtctttctttccccaggccgagacatgcaaggtgcagagcaaccgggtgttctgtgacacaatgaattctctgaccctgcctagcgaggtgaacctgtgcaatgtggacatctttaatccaaagtacgattgtaagatcatgaccagcaagacagatgtgagctcctctgtgatcacaagcctgggcgccatcgtgtcctgctacggcaagaccaagtgtacagcctccaacaagaatagaggcatcatcaagaccttctccaacggctgcgactacgtgagcaataagggcgtggatacagtgtctgtgggcaacaccctgtactatgtgaataagcaggagggcaagagcctgtacgtgaagggcgagccaatcatcaacttctatgaccccctggtgttcccttccgacgagtttgatgccagcatctcccaggtgaacgagaagatcaatcagagcctggcctttatcaggaagtccgatgagctgctgcacaacgtgaatgccgtgaagagcaccacaaacatcatgatcaccacaatcatcatcgtgatcatcgtgatcctgctgagcctgatcgccgtgggcctgctgctgtactgtaaggcccgctccacccccgtgacactgtccaaggatcagctgtctggcatcaacaatatcgcctttagcaactaaagatctttttccctctgccaaaaattatggggacatcatgaagccccttgagcatctgacttctggctaataaaggaaatttattttcattgcaatagtgtgttggaattttttgtgtctctcactcggaaggacataagggcggccgctagc

SEQ ID NO: 5 - protein, 574 aa, synthetic construct
  REGION 1..574: note = Synthetic: The full-length RSV A2 strain pre-fusion F gene (DS-Cav1) using humanized codons for expression is 574 aa long.
MELLILKANAITTILTAVTFCFASGQNITEEFYQSTCSAVSKGYLSALRTGWYTSVITIELSNIEENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHHVNAGKSTTNIMITTIIIVIIVILLSLIAVGLLLYCKARSTPVTLSKDQLSGINNIAFSN

SEQ ID NO: 6 - protein, 513 aa, synthetic construct
  REGION 1..513: note = Synthetic: RSV A2 strain pre-fusion truncated F (DS-Cav1) 513 aa protein without transmembrane domain and cytoplasmic tail, which was formulated with adjuvants. 9 aa fusion peptide was not deleted
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 7 - DNA, 1737 bp, synthetic construct
  misc_feature 1..1737: note = Synthetic: codon optimized nucleotide sequence of RSV-F-A2
  source 1..1737: mol_type = other DNA
gtcgacatggagctgctgatcctgaaggccaacgccatcaccacaatcctgaccgccgtgacattctgctttgcctccggccagaatatcacagaggagttctaccagtctacctgtagcgccgtgtccaagggctacctgtctgccctgaggaccggctggtatacatctgtgatcaccatcgagctgagcaacatcaaggtgaacaagtgcaatggcacagacgccaaggtgaagctgatcaagcaggagctggataagtacaagaatgccgtgaccgagctgcagctgctgatgcagtctaccccagccacaaacaatcgggcccggagagagctgcccagattcatgaactataccctgaacaatgccaagaagaccaatgtgacactgagcaagaagcgcaagaggcgcttcctgggctttctgctgggagtgggatctgccatcgcaagcggagtggccgtgtgcaaggtgctgcacctggagggcgaggtgaacaagatcaagtccgccctgctgtctacaaacaaggccgtggtgagcctgtccaatggcgtgtccgtgctgacctttaaggtgctggacctgaagaattatatcgataagcagctgctgcctatcctgaacaagcagtcttgtagcatctccaatatcgagacagtgatcgagttccagcagaagaacaataggctgctggagatcacccgcgagttttctgtgaacgccggcgtgaccacacccgtgagcacatacatgctgaccaatagcgagctgctgtccctgatcaacgacatgcctatcaccaatgatcagaagaagctgatgtccaacaatgtgcagatcgtgcggcagcagtcttatagcatcatgtgcatcatcaaggaggaggtgctggcctacgtggtgcagctgccactgtatggcgtgatcgacacaccctgctggaagctgcacacctcccctctgtgcaccacaaacaccaaggagggctctaatatctgcctgacccggacagacagaggctggtactgtgataacgccggctccgtgtctttctttccccaggccgagacatgcaaggtgcagagcaaccgggtgttctgtgacacaatgaattctctgaccctgcctagcgaggtgaacctgtgcaatgtggacatctttaatccaaagtacgattgtaagatcatgaccagcaagacagatgtgagctcctctgtgatcacaagcctgggcgccatcgtgtcctgctacggcaagaccaagtgtacagcctccaacaagaatagaggcatcatcaagaccttctccaacggctgcgactacgtgagcaataagggcgtggatacagtgtctgtgggcaacaccctgtactatgtgaataagcaggagggcaagagcctgtacgtgaagggcgagccaatcatcaacttctatgaccccctggtgttcccttccgacgagtttgatgccagcatctcccaggtgaacgagaagatcaatcagagcctggcctttatcaggaagtccgatgagctgctgcacaacgtgaatgccgtgaagagcaccacaaacatcatgatcaccacaatcatcatcgtgatcatcgtgatcctgctgagcctgatcgccgtgggcctgctgctgtactgtaaggcccgctccacccccgtgacactgtccaaggatcagctgtctggcatcaacaatatcgcctttagcaactaaagatct